(19) United States
(12) Patent Application Publication
(10) Pub. No.: US 2021/0130291 A1
(43) Pub. Date: May 6, 2021

(54) METHODS FOR TREATING DIABETES USING VDAC1 INHIBITORS

(71) Applicants: Albert SALEHI, Malmo (SE); Claes B. WOLLHEIM, Malmo (SE); THE NATIONAL INSTITUTE FOR BIOTECHNOLOGY IN THE NEGEV LTD., Beer Sheva (IL)

(72) Inventors: Albert SALEHI, Malmo (SE); Claes B. WOLLHEIM, Malmo (SE); Varda SHOSHAN-BARMATZ, Orner (IL)

(21) Appl. No.: 16/472,609

(22) PCT Filed: Dec. 21, 2017
(86) PCT No.: PCT/IL2017/051379

§ 371 Date: Jun. 21, 2019

Related U.S. Application Data
(60) Provisional application No. 62/437,703, filed on Dec. 22, 2016.

(51) Int. Cl. C07D 207/273; C07D 401/04; A61K 45/06; A61P 3/10

(52) U.S. Cl. CPC C07D 207/273; A61P 3/10; A61K 45/06; C07D 401/04

(57) ABSTRACT

The present invention relates to compositions and methods for treating prediabetes and diabetes and delaying progression of the disease. The present invention uses molecules that specifically bind to and inhibit Voltage Dependent Anion Channel (VDAC1) that is expressed on the beta cells of diabetic subjects. Particularly, the present invention discloses the use of substituted piperazine and piperidine derivatives as specific inhibitors of VDAC1 for preventing the progression of and treating prediabetes and diabetes.

### FIELD OF THE INVENTION

The present invention relates to compositions and methods for treating prediabetes and diabetes and delaying progression of the disease. Particularly, the present invention discloses the use of substituted piperazine and piperidine derivatives as specific inhibitors of VDAC1 for preventing the progression of and treating prediabetes and diabetes.

### BACKGROUND OF THE INVENTION

Diabetes is a severe metabolic disease that shortens life expectancy through cardiovascular and chronic kidney diseases, as well as causing peripheral nerve diseases and blindness. Approximately 12% of global health costs are spent on diabetes. Diabetes is due to either the pancreas not producing enough insulin or the cells of the body not responding properly to the insulin produced. There are two main types of diabetes;

Type 1 Diabetes Mellitus (T1D) is one of the most common multifactorial endocrine and metabolic diseases in childhood resulting in persistent hyperglycemia. To date, approximately 490,000 children have been diagnosed with T1D and some 78,000 children under the age of 15 are estimated to develop T1D annually worldwide.

T1D is classified as either immune-mediated or idiopathic (of unknown etiology) diabetes. The most common form of T1D in Western societies is immune-mediated which results from the breakdown of beta-cell-specific self-tolerance by T-lymphocytes.

Existing treatments for T1D are primarily focused on insulin supplementation. However, despite the beneficial effects of life-long insulin therapy on glucose homeostasis, it unfortunately does not eliminate severe diabetic complications such as retinopathy and nephropathy as well as cardiovascular and cerebrovascular diseases Moreover, T1D patients experience frequent episodes of hypoglycemia, when insulin injections are not adapted to the nutritional state or during exercise, which is thought to have long-term consequences on retinal and brain function.

Several studies have reported that residual beta-cells (β cells) remain in the pancreatic islets of T1D patients. In fact, up to 40-50% of beta-cells may escape destruction, but insulin secretion is insufficient to maintain normal blood glucose regulation. In vitro culture of islets from T1D subjects at physiological glucose concentration has shown time-dependent improvement of glucose-stimulated insulin secretion (Lupi R, et al. 2004. Diabetes/metabolism research and reviews, 20(3), 246-251; Krogvold L et al. 2015. Diabetes 64:2506-2512). These results suggest that the residual beta-cells are dysfunctional in vivo but can resume regulated insulin secretion after extraction from the harmful environment.

Type 2 diabetes (T2D) is a world-wide health problem in the wake of the obesity epidemic. Approximately 400 million people world-wide suffer from Type 2 diabetes and 320 million are estimated to have pre-diabetes (Zimmet P and Alberti K G. 2016. Nature Rev Endo 10:616-622). Normoglycemia is maintained when obesity-associated insulin resistance is compensated by increased insulin secretion.

T2D has a strong genetic component and most frequently occurs when members of diabetes-prone families cumulate risk factors, such as obesity, smoking, repeated pregnancies or shift work. It appears that the disease occurs as a sequel of obesity, when the insulin secretion from the pancreatic beta cells cannot adapt to the increased insulin requirements of the organs resistant to the hormone. T2D develops after years of impaired glucose tolerance (IGT) (Ligthart S, et al. 2016. Lancet Diabetes & endocrinology 4:44-51). Elevated average blood glucose concentrations exert harmful effects on the beta cells, so called glucotoxicity (Weir G C et al. 2004. Diabetes 53, Suppl 3: 516-21).

Glucotoxicity contributes to β cell decompensation, a phenomenon which also exists in healthy subjects as glucose infusion attenuates glucose-stimulated insulin secretion (GSIS). The overt T2D with hyperglycemia, increased urine volume and thirst is preceded by a period of prediabetes, often lasting up to 7 years. Prediabetes is defined as elevated fasting blood glucose or impaired lowering of blood glucose after an oral glucose challenge. T2D is reversible early after onset of the disease by life-style interventions aiming at weight loss and implementing physical exercise (Al-Mrabeh et al., 2016. Diabetologia 59:1753-1759). Such interventions show, however, low patient compliance.

In healthy cells, Voltage-dependent anion channel (VDAC), a multi-functional protein, is positioned at the crossroad of metabolic and survival pathways considered a master-gatekeeper regulating the flux of metabolites and ions between mitochondria and the cytosol. Of the three VDAC isoforms, VDAC1 and VDAC2 mediate ADP/ATP exchange and calcium flux in mitochondria, while VDAC3 function is less clear (Shoshan-Barmatz V et al., 2015; Biochim. Biophys. Acta 1848:2547-2575; Shoshan-Barmatz V et al., 2010. Molecular aspects of medicine 31:227-285).

VDAC has also been localized to cell compartments other than mitochondria, such as the plasma membrane of various cells, the sarcoplasmic reticulum of skeletal muscles, the endoplasmic reticulum (ER) of rat cerebellum, and in synaptosomes from the Torpedo electric organ. Immunofluorescence analysis revealed the presence of VDAC in various cell surfaces, such as the membranes of lymphocytes, epithelial cells and astrocytes. Flow cytometry and EM immunogold labeling of a post-synaptic membrane fraction from brain also detected VDAC in plasma membrane (De Pinto V et al., FEBS Lett. 2010. 584:1793-1799.). Such plasmalemmal (pl)-VDAC1 (also referred to as plasma membrane VDAC) contains a hydrophobic leader sequence (Buettner R et al., Proc Natl Acad Sci USA. 2000. 97:3201-3206).

VDAC1 is the sole channel located at the outer mitochondrial membrane (OMM) mediating metabolic cross-talk between mitochondria and the cytosol, transporting metabolites, ions, nucleotides, Ca²⁺ and more, thus regulating mitochondrial activity. VDAC1 also plays a key role in apoptosis, participating in the release of apoptotic factors from mitochondria and interacting with anti-apoptotic regulators (Shoshan-Barmatz et al., 2015, ibid; Shoshan-Barmatz et al., 2010, ibid). Mitochondrial proteome analysis reveals altered expression of VDAC and multiple other proteins involved in nutrient metabolism, ATP synthesis, cellular defense, glycoprotein folding and mitochondrial DNA stability in clonal pancreatic β-cells exposed to high glucose (Ahmed et al., 2010. Islets, 2(5):283-292). Under glucotoxic condition, the expression of VDAC1 was upregulated while that of VDAC2 was downregulated.

An inventor of the present invention and co-worker have developed a novel group of piperazine- and piperidine-based compounds, which directly interact with and have high inhibitory activity of VDAC transport activity, oligomerization and other activities associated with mitochondria dysfunction as changes in the level of intracellular Ca²⁺, mitochondria membrane potential and reactive oxygen species (ROS). These derivatives are thus useful as inhibitors of its channel conductance, its oligomerization and thereby as inhibitors of the release of apoptogenic proteins from the mitochondria, as well as inhibitors of apoptotic cell death or other cell death types as necrosis (International Application Publication No. WO 2017/046794; Ben Hail et al., 2016. J Biol. Chem. 291(48):24986-25003).

U.S. Patent Application Publication No. 2011/0020312 discloses methods and formulations for treating metabolic disorders (such as diabetes) in humans using epimetabolic shifters, multidimensional intracellular molecules or environmental influencers.

U.S. Pat. No. 6,140,067 discloses diagnostic methods for early detection of a risk for developing Type 2 diabetes mellitus in humans, and screening assays for therapeutic agents useful in the treatment of Type 2 diabetes mellitus, by comparing the levels of one or more indicators of altered mitochondrial function. Indicators of altered mitochondrial function include enzymes such as mitochondrial enzymes and ATP biosynthesis factors. Other indicators of altered mitochondrial function include mitochondrial mass, mitochondrial number and mitochondrial DNA content, cellular responses to elevated intracellular calcium and to apoptogens, and free radical production.

There is still an unmet need for, and it would be highly advantageous to have novel methods for treating diabetes, particularly for protecting beta cells through the preservation of their function and thereby preventing or inhibiting the progress of pre-diabetes conditions to Type-2 diabetes.

### SUMMARY OF THE INVENTION

The present invention relates to the use of molecules capable of specifically binding to and inhibiting the Voltage-Dependent Anion Channel Type 1 (VDAC1) protein for the treatment of diabetes. The VDAC1 inhibitory molecules are capable of reducing the VDAC1 channel conductance and inhibiting VDAC1-mediated metabolite transport, particularly inhibiting VDAC1 molecules of the plasma membrane of pancreatic β-cells. According to one aspect the compounds capable of specifically interacting with and inhibiting VDAC1 are small organic compounds.

According to another aspect the compounds of the invention are useful in prevention of the progression of prediabetes to diabetes and in treating these conditions.

The present invention is based in part on the unexpected discovery that in the β-cells of diabetic subjects VDAC1 is not only overexpressed but actually appears within the plasma membrane. This renders VDAC1 accessible to inhibition by various specific inhibitors, including small organic molecules, antibodies and peptides. It is now disclosed for the first time that intervention using specific inhibitors of VDAC1 can restore β-cell function and thereby treat diabetes or prevent progression of the disease.

The present invention is additionally based in part on the unexpected discovery that compounds of general formula (I¹) directly bind to and have high inhibitory effect on VDAC1 conductance and metabolite transport activity, particularly on ATP and Ca²⁺ transport. The compounds, particularly molecules designated herein VBIT-4 and AKOS (also designated AKOS022075291) attenuate the loss of ATP through VDAC1 in β-cells isolated from diabetic donors, and restored ATP generation and glucose-induced insulin secretion. Furthermore, administration of VBIT-4 to diabetic (db/db) mice prevented the development of hyperglycemia and enhanced the glucose-stimulated insulin secretion in the treated mice.

The present invention shows for the first time that islet cells isolated from donors diagnosed for Type 2 Diabetes (T2D) over-expressed VDAC Type 1 (VDAC1) while the expression of VDAC Type 2 (VDAC2) was down-regulated. Without wishing to be bound by any theory or mechanism of action, VDAC1 over-expression leads to translocation of VDAC1 into the plasma membrane of islet cells resulting in impaired reductive capacity and loss of the essential metabolic coupling factor ATP associated with reduction in glucose-stimulated insulin secretion (GSIS) and diabetes. Thus, therapeutic concentrations of compounds of general Formula (I¹) restore insulin secretion and prevent hyperglycemia in diabetic mice by direct inhibition of VDAC1 aberrantly expressed in the β-cell membrane.

In a broad aspect, the present invention provides a method of treating diabetes and/or preventing the progress of diabetes in a subject in need thereof, the method comprises administering to the subject a therapeutically effective amount of a compound binding to and inhibiting VDAC1. In particular, such compound inhibits VDAC1 expressed on pancreatic beta-cells. According to certain embodiments, the VDAC1 is expressed on the surface of the pancreatic beta-cells.

In another aspect, the present invention provides a method of restoring dysfunctional beta-cells, the method comprising contacting the dysfunctional beta-cells with a compound binding to and inhibiting VDAC 1 expressed on pancreatic beta-cells in a subject.

According to one aspect, the present invention provides a method of treating and/or preventing the progress of diabetes in a subject in need thereof, the method comprises administering to the subject a therapeutically effective amount of at least one compound of general Formula (I¹), wherein:

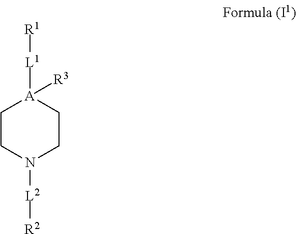

Wherein

A is carbon (C) or nitrogen (N);

R³ is absent, or is selected from a hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen; wherein when A is nitrogen (N), R³ is absent;

L¹ is absent or is an amino a linking group —NR₄—, wherein R⁴ is hydrogen, a C₁₋₅-alkyl, a C₁₋₅-alkylene or a substituted alkyl —CH₂—R, wherein R is a functional group selected from the group consisting of hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl;

R¹ is an aromatic moiety which is optionally substituted with one or more of Z;

Z is independently at each occurrence a functional group selected from the group consisting of hydrogen, halo, haloalkyl, haloalkoxy, perhaloalkoxy or C₁₋₂-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl;

L² is a linking group, such that when A is nitrogen (N), L² is a group consisting of 4-10 atoms, apart from hydrogen atoms, optionally forming a ring, whereof at least one of the atoms is nitrogen, said nitrogen forming part of an amide group; and when A is carbon (C), then L² is selected from C₁₋₄ alkylene, or a group consisting of 4-10 atoms, apart from hydrogen atoms, optionally forming a ring, whereof at least one of the atoms is nitrogen, said nitrogen forming part of an amide group; and

R² is a phenyl or a naphthyl, optionally substituted with halogen,

or an enantiomer, diastereomer, mixture or salt thereof.

According to additional aspects, the present invention provides a compound of the general Formula (I¹) for use in treating and/or preventing the progress of diabetes in a subject in need thereof.

According to certain embodiments, the compound of general Formula I¹ is selected from the group consisting of a compound of general Formulae (Ia), (Ib), (Ic), and (Id) as described hereinafter. Each possibility represents a separate embodiment of the present invention. According to certain exemplary embodiments, the compound is selected from the group consisting of a compound of structural Formulae 1, 2, 3, 4, 5, 6, 7, 8 and 9 as described hereinafter. Each possibility represents a separate embodiment of the present invention.

According to certain currently preferred exemplary embodiments the compound is N-(4-chlorophenyl)-4-hydroxy-3-(4-(4-(trifluorometh¬oxy)phen¬yl)-piperazin-1-yl)butanamide (Formula 1), designated herein VIBT-4. According to some embodiments, the racemic mixture of compound VBIT-4 is used. According to some embodiments, an optically pure (+) enantiomer of VBIT-4 is used. According to some embodiments, an optically pure (−) enantiomer of VBIT-4 is used.

According to certain embodiments the compounds used in the methods of the present invention are compounds having Formula (IIa) as described hereinafter.

According to certain embodiments, the compound is of the general formula (IIa):

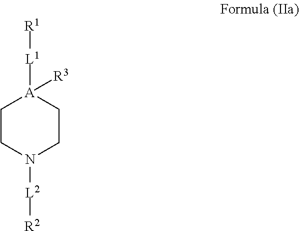

wherein:
A is carbon (C);

R³ is hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen;

L¹ is a linking group which is an amino linking group —NR⁴—, wherein R⁴ is hydrogen, a C₁₋₅-alkyl, a C₁₋₅-alkylene or a substituted alkyl —CH₂—R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl or heteroaryl; when R³ is hydrogen, then L¹ is —NR⁴—, preferably —NH—; when R³ is heteroalkyl group comprising 3-12 atoms, then L¹ is preferably —NCₙH₂ₙ—, such that it forms a ring with R³;

R¹ is an aromatic moiety, which is optionally substituted with one or more of C₁₋₂-alkoxy, e.g. haloalkoxy, such as C₁₋₂-perfluoroalkoxy;

L² is a linking group consisting of 4-10 atoms (apart from hydrogen atoms), optionally forming a closed ring, whereof at least one of the atoms is nitrogen, said nitrogen forming part of an amide group or L² is C₁₋₅ alkyl or C₁₋₅ alkylene; said linking group L² bonds piperidine or piperazine moiety at nitrogen (N) atom; typically, L² is selected from the group consisting of butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methylbutanamidylene, 2-pyrrolidonylene, pyrrolidine-2,5-dionylene, 5-thioxo-2-pyrrolidinonylene and 5-methoxy-2-pyrrolidinonylene; and

R² is an aryl, optionally substituted with halogen, optionally when R² is a phenyl it is substituted with halogen, further optionally when R² is naphthyl, L² is an alkylenyl group. In a specific embodiment, R³ is hydrogen, L¹ is —NH—, and R¹ is a phenyl substituted with trifluoromethoxy,

or an enantiomer, diastereomer, mixture or salt thereof.

According to certain exemplary embodiments, the compound according to general Formula (IIa) is 1-(4-Chloro-phenyl)-3-[4-(4-trifluoromethoxy-phenylamino)-piperidin-1-yl]-pyrrolidine-2,5-dione having the structural Formula 10 (AKOS). According to other exemplary embodiments, the compound according to general Formula (IIa) has the structural Formula 11 as described hereinafter.

The invention also relates to the stereoisomers, enantiomers, mixtures thereof and salts thereof, of the compounds of general Formula I¹ and general Formula IIa and all their derivatives, according to the invention.

According to additional aspects, the use of the compounds of the present invention comprises preventing the progression of pre-diabetes to diabetes.

According to certain embodiments, the use of the compounds of the present invention for treating diabetes comprises inducing glucose-stimulated insulin secretion.

According to certain embodiments, the use of the compounds of the present invention for treating diabetes comprises improving glucose tolerance.

According to certain embodiments, the use of the compounds of the present invention for treating diabetes comprises restoring insulin secretion from pancreatic β-cells of the subject.

According to certain embodiments, the use of the compounds of the present invention for treating diabetes comprises prevention of β-cell dysfunction.

According to certain embodiments, the subject to be treated with the compounds of the present invention is selected from the group consisting of a subject having prediabetes and a subject having diabetes. According to certain embodiments the subject having diabetes is newly diagnosed early after onset of the disease. According to certain embodiments, the subject has severe hyperglycemia. According to certain embodiments the diabetes is selected from the group consisting of diabetes Type 1 and diabetes Type 2.

According to additional embodiments, the subject shows symptoms of pre-diabetes. According to other embodiments, the subject is a member of a family with strong genetically determined propensity for diabetes Type 2. According to these embodiments, the diabetes in diabetes Type 2.

According to certain embodiments, the subject is a human. The human subject can be at any age, including pre-pubertal child, post-pubertal child, adolescent and adult.

The VDAC inhibitory compounds of the present invention can be administered to the subject in need thereof by any suitable route of administration as is known to a person skilled in the art.

According certain embodiments, the compounds of the present invention are administered to the subject in need thereof within a pharmaceutical composition further comprising a pharmaceutically acceptable excipient, diluent or carrier.

The pharmaceutical compositions of the present invention can be formulated for administration by a variety of routes including oral, parenteral, transdermal, topical, intranasal, or via a suppository.

According to certain exemplary embodiments, the pharmaceutical composition comprising the compounds of the invention is formulated for oral administration.

According to certain exemplary embodiments, the pharmaceutical composition comprising the compounds of the invention is formulated for parenteral administration.

According to some embodiments, the pharmaceutical composition further comprises at least one additional active agent.

According to additional aspect, the present invention provides a compound specifically binding to and inhibiting VDAC1 for use in treating diabetes and/or preventing the progress of diabetes in a subject in need thereof. According to certain embodiments, the VDAC1 is expressed on pancreatic β-cells. According to certain embodiments, the VDCA1 is expressed on the surface of the pancreatic β-cells. In a further embodiment diabetes is type I. In another embodiment diabetes is type II. In a still further embodiment diabetes is non-insulin dependent. In a further embodiment diabetes is insulin dependent.

According to certain embodiments, the compound specifically binds to and inhibits VDAC1 expressed on pancreatic β-cells.

According to certain embodiments, the compound inhibits ATP transport via the VDAC1.

According to certain embodiments, the compound does not significantly bind to and/or inhibit VDAC2.

According to certain embodiments, the compound is selected from the group consisting of a small organic molecule, a peptide and an antibody.

In a further embodiment the small organic molecule is less than 900 Da. Typically, such small organic molecule comprises a 5- or 6-membered heterocycle containing at least one heteroatom selected from N, S, and O. In some embodiments the heteroatom is selected from N and O such as a piperazine and/or piperidine ring. Moreover, in a further embodiment the small organic molecule comprises a 5- or 6-membered heterocycle containing at least one heteroatom selected from N, S, and O, wherein the heterocycle is linked to an aromatic ring or a heteroaromatic ring, such as two aromatic rings, or two heteroaromatic rings or one aromatic ring and one heteroaromatic ring.

According to certain exemplary embodiments, the compound is an antibody. Typically the antibody is a monoclonal antibody, such as a recombinant antibody. The antibody is selected from a mammalian antibody, a human antibody, and a humanized antibody. The antibody may be a fragment or a full antibody, as long as it comprises at least one VDAC1 binding site. In some embodiments the fragment of the antibody is selected from F(ab), F(ab)₂, Fv or single chain Fv, which retains the binding activity to VDAC1. In some embodiments the antibody or antibody fragment is specific for VDAC1 and does not bind to other VDAC isoforms.

According to certain embodiments, preventing the progression of diabetes comprises preventing progression of prediabetes to diabetes.

According to certain embodiments, preventing the progression of diabetes comprises preventing progression of non-insulin dependent diabetes to insulin dependent diabetes.

According to certain embodiments, treating diabetes comprises at least one of inducing glucose-stimulated insulin secretion; improving glucose tolerance; restoring insulin secretion from pancreatic β-cells of a subject affected with diabetes; and prevention of β-cell dysfunction.

According to yet further aspects, the present invention provides a piperazine and/or piperidine derivative specifically binding to and inhibiting VDAC1 for use in treating diabetes and/or preventing the progress of diabetes in a subject in need thereof.

According to certain embodiments, the piperazine and/or piperidine derivative specifically binds to and inhibits VDAC1 expressed on pancreatic β-cells. According to certain embodiments the VDAC1 is expressed on the surface of the pancreatic β-cells.

According to certain embodiments, the piperazine and/or piperidine derivative inhibits ATP transport via VDAC1.

According to certain embodiments, the piperazine and/or piperidine derivative does not significantly bind to and/or inhibit VDAC2.

According to certain embodiments, preventing the progression of diabetes comprises preventing progression of prediabetes to diabetes.

According to certain embodiments, treating diabetes comprises at least one of inducing glucose-stimulated insulin secretion; improving glucose tolerance; restoring insulin secretion from pancreatic β-cells of a subject affected with diabetes; and prevention of β-cell dysfunction.

In a further aspect, the present invention relates to an oral pharmaceutical composition comprising a compound of general Formula (I¹). In some embodiments the oral pharmaceutical composition comprises a compound of general Formula (I). In specific embodiments the compound is selected from structural Formula 1, 2, 3, 4, 5, 6, 7, 8, 9. According to some embodiments the oral pharmaceutical composition further comprises a pharmaceutically acceptable carrier and/or excipient.

In an embodiment the oral composition is selected from a solid or liquid composition.

In a further embodiment the oral composition comprises a dosage of from 1 mg to 700 mg per dosage unit. In some embodiments the oral composition comprises a dosage of from 10 mg to 500 mg. In further embodiments the oral composition provides from 20 to 250 mg per dosage unit.

According to additional aspect, the present invention provides a method of screening for a molecule useful in treating diabetes, the method comprising;
(a) providing a cell culture comprising cells expressing VDAC1 localized in the cell plasma membrane;
(b) exposing the cells to a candidate molecule;
(c) measuring extracellular ATP amount within the culture medium and optimally intracellular ATP amount within a portion of lysed cells; and
(d) comparing the extracellular ATP amount and optionally the intracellular ATP amount to the amounts measured in a corresponding cell culture not exposed to the candidate molecule or to a predetermined control values;
wherein a reduction in the extracellular ATP amount and/or an increase in the intracellular ATP content in the cell culture exposed to said candidate compared to the corresponding cell culture or predetermined control values identifies said molecule as useful in treating diabetes.

According to certain embodiments, the extracellular ATP amount within the medium and optionally the intracellular ATP amount are measured after incubating the cell culture with the candidate molecule for from about 5 min to about 5 h. According to certain embodiments, the incubation time is from about 1 min to 2.5 h. According to certain exemplary embodiments, the ATP amount is measured after incubation of the cell culture with the candidate molecule for from about 15 min. to about 1 h.

According to certain embodiments, the cells are pancreatic β-cells or clonal derivatives thereof.

Any method as is known in the Art for measuring ATP content within a medium can be used with the method of the invention.

Compounds identified by the screening method of the invention are contemplated within the scope of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1a-1l demonstrate VDAC expression in β-cells. FIG. 1a: mRNA expression of VDAC1 and VDAC2 analyzed by qPCR in islets from non-diabetic (ND) and T2D donors. Data are expressed as mean±SEM of 19 ND and 18 T2D donors. ***p<0.001. FIG. 1b: Correlation between islet VDAC1 mRNA and preterminal blood glucose reflected by HbA1c in ND donors ***p<0.001. FIG. 1c: Expression of VDAC1 and VDAC2 mRNA analyzed by qPCR in islets from ND donors cultured at physiological glucose (5 mM), 5G, or glucotoxic condition (20 mM), 20G, for 72 h. Data are mean±SEM of 5 donors. ***p<0.001. FIG. 1d: Expression of VDAC1 in non-diabetic human islets cultured for 72 h at 5 and 20 mM glucose in the presence or absence of metformin (Met, 20 μM). FIG. 1e: Both ChREBP and TXNIP mRNA is increased in islets from T2D donors. Mean±SEM of 4-5 donors. *p<0.05, ***p<0.001. FIG. 1f: Glucose-induced (20 mM) VDAC1 expression is blunted after knock-down of either ChREBP or TXNIP in INS-1 cells. Data are expressed as mean±SEM of 5 independent experiments). **p<0.01, ***p<0.001. FIGS. 1g-1h show representative Western blots and fold change by densitometry normalized to β-actin for VDAC1 (FIG. 1g) and VDAC2 (FIG. 1h) in islet extracts from non-diabetic (ND) and T2D donors (n=5 each). ***p<0.001. FIG. 1i-1l show VDAC1 and VDAC2 transcript levels after overexpression (OE) of VDAC1 (FIG. 1i) and knock-down (KD) of VDAC2 (FIG. 1j) in INS-1 cells. The transcripts were analyzed by qPCR. Results are mean+SEM of 3 independent experiments with 4 technical replicates. FIG. 1k-1l, reciprocal changes in VDAC mRNA levels after overexpression of VDAC1 or knockdown of VDAC2 in INS-1 cells. VDAC1 expression is shown in (k) and VDAC2 expression in (1). Control (Ctrl) empty plasmid for VDAC1 OE (FIG. 1k, 1l) and Scramble RNA (Scr) for VDAC2 (FIG. 1i, 1j). Data are expressed as mean±SEM (n=9 different experiments). **p<0.01, ***p<0.001.

FIGS. 2a-2m demonstrate VDAC function in β-cells. FIG. 2a-2b: Glucose-stimulated insulin secretion (GSIS) in INS-1 cells after overexpression of VDAC1 or knock-down of VDAC2. VDAC1 overexpression (OE) (FIG. 2a) or VDAC2 knock-down (KD) (FIG. 2b) reduces glucose-stimulated insulin secretion (GSIS) in INS-1 cells incubated at 2.8 or 16.7 mM glucose for 60 min. Controls were empty plasmid (FIG. 2a) and scramble (FIG. 2b). FIG. 2c: Oxygen consumption rate (OCR) in INS-1 cells with VDAC1 overexpression or VDAC2 knock-down (KD) at basal (2.8 mM) and glucose-stimulated (16.7 mM) conditions. The data are mean±SEM of five independent experiments. *p<0.05, **p<0.01. FIG. 2d: Effect of VDAC1 or VDAC2 knockdown (KD) on ATP content of islets cultured at 5 or 20 mM glucose (72 h) incubated at 1 or 16.7 mM glucose for 1 h. Mean±SEM of 3 donors. FIG. 2e: Insulin secretion for the same islets as in (d). FIG. 2f: Cellular reductive capacity and effect of VDAC knockdown in human islets under glucotoxic condition. VDAC1 KD protects human islet cells from glucotoxicity induced decrease in cellular reductive capacity (formazone production) while VDAC2 KD is harmful. Islets from 5 donors (used in separate experiments) were cultured at either 5 or 20 mM glucose for 72 h. Mean±SEM. FIG. 2g: Oxygen consumption rate (OCR) in INS-1 cells after overexpression of VDAC1 (•) or knock-down of VDAC2 (º). Both basal (2.8 mM glucose, 2.8G) and glucose-stimulated (16.7 mM glucose, 16.7G) increase in OCR were decreased after alteration of VDAC expression. The data are mean±SEM of five different experiments. Subsequent additions were as follows: oligomycin (Olig, an inhibitor of ATP synthase) (0.4 μM), dinitrophenol (DNP, an uncoupler) (0.4 μM) and rotenone (Rot, an inhibitor of complex I) (0.1 μM). FIG. 2h: Both basal and glucose-stimulated increase in OCR is attenuated in INS-1 cells after culture at 20 mM glucose (20G) for 72 h compared to 5 mM glucose (5G) culture. The data are mean±SEM of five different experiments. Additions of oligomycin, dinitrophenol or rotenone as under (g). FIG. 2i, Average data of the OCR experiments. *p<0.05, **p<0.01. FIG. 2j-2l: demonstrate mitochondrial (Mito) and cytosolic (Cyto) Ca²⁺ in INS-1 cells cultured at 5 or 20 mM glucose as well as after overexpression (OE) of VDAC1 or knock-down (KD) of VDAC2. FIG. 2j: representative traces of mito Ca²⁺ under control (5G, 72 h) and glucotoxic (20G, 72 h) conditions with addition of indicated stimuli (20 mM glucose and 70 mM K+). FIG. 2k: Mito Ca²⁺ as area under the curve (AUC) after acute stimulation with 20 mM glucose. FIG. 2l, Cyto Ca²⁺ expressed as AUC after acute stimulation with 20 mM glucose. Data are mean±SEM. Mito and Cyto Ca²⁺ were monitored simultaneously in respectively 30 cells (5 mM glucose culture), 28 cells (20 mM) culture, 27 cells with VDAC1 OE and 29 cells after VDAC2 KD. *p<0.05, **p<0.01. FIG. 2m: shows the effects of VDAC1 overexpression (OE) or VDAC2 knock-down (KD) on INS-1 cell apoptosis. Experiments were performed either directly after 48 h of culture in 5 mM glucose or after an additional 72 h culture in 20 mM glucose. Results are from three independent experiments performed with 2-3 technical replicates.

FIGS. 3a-3f show the localization of VDAC1 in β-cells. FIG. 3a: Representative immunofluorescence images of VDAC1 and VDAC2 in human islet β-cells from non-diabetic (ND) and T2D donors, one of which had received metformin therapy. Note VDAC1 expressed prominently on the β-cell surface in T2D islets. FIG. 3b: β-cell surface expression of VDAC1 given as ratio of surface/cytosolic VDAC1 immunofluorescence intensity in β-cells of ND or T2D (n=8 donors each) and the one with metformin-therapy. FIG. 3c: Correlation between VDAC1-cell surface expression and HbA1c values in 15 islet donors. FIG. 3d: Co-localization of VDAC1 with SNAP25 was examined by double immunostaining in insulin-positive cells in pancreatic sections from ND and T2D donors. Calculation of coefficience (VDAC1/SNAP25) was performed by confocal image analyzer software Zen2012. Mean±SEM of 9 sections from each donor were analyzed (3 donors each group). Bar indicates 5 um. *p<0.05, **p<0.01 FIG. 3e: demonstrates that glucotoxicity causes cell surface expression of VDAC1. The ratio (Surface to Cytosol) expression of VDAC1 is increased after culturing human islets at 20 mM glucose (20G) for 72 h. Data are expressed as mean±SEM from 4 organ donors. *P<0.05. FIG. 3f: INS-1 cells were cultured at 5 (5G) or 20 mM glucose (20G) for 72 h and then immunostained for VDAC1. Translocation of VDAC1 from intracellular sites to the cell membrane was calculated. **p<0.01 (n=7 replicates in each group from 3 independent experiments).

FIGS. 4a-4m demonstrate that VDAC1 cell surface expression alters INS-1 cell ATP handling, insulin secretion and membrane conductance. FIG. 4a: ATP release after 1 h incubation at 1 or 16.7 mM glucose from INS-1 cells transfected with either mitochondrial VDAC1 (mtVDAC1) or plasma membrane targeted VDAC1 (pVDAC1) and control cells. Mean±SEM (n=4 different experiments). FIG. 4b: Glucose-stimulated insulin secretion measured in the same experiments as in (a). FIG. 4c: Effect of VDAC1-antibody (VDAC1-ab, 10 nM), metformin or the VDAC1 blockers AKOS022075291 and VBIT-4 (20 uM each) on ATP release after 1 h incubation at 1 mM glucose from INS-1 cells transfected with control plasmid or plVDAC1. Mean±SEM from at least 3 independent experiments. FIG. 4d: Membrane conductance recorded by the whole cell patch-clamp technique in INS-1 cells overexpressing mtVDAC1 or plVDAC1. Mean±SEM of 10 cells each. FIG. 4e: Ratio of membrane conductance after and before acute additions of extracellular solution in INS-1 cells overexpressing plVDAC1, without and with either VDAC1-ab or metformin. Mean±SEM of 11 cells in each group are shown. FIG. 4f: Membrane conductance (whole-cell patch-clamp) in control and plVDAC1-transfected INS-1 cells in the presence or absence of metformin (20 uM) within 1 hour. Mean±SEM of 15 cells in each group are shown. FIG. 4g: Metformin (30 uM) reduces conductance of planar lipid bilayers reconstituted with VDAC1. Average steady-state conductance measured at the indicated voltage, before (•) and 10-30 min after metformin addition (º). Mean±SEM of 3 independent measurements, in the figure top a representative trace at 10 mV is shown. FIG. 4h: Same as in (e) using VBIT-4 (20 uM). *p<0.05, **p<0.01. FIG. 4i: metformin-inhibited (Met) ATP release in plVDAC1 transfected INS-1 cells is neither attenuated by an AMPK inhibitor (MRT199665, 5 μM) (MRT) nor mimicked by the antioxidants resveratrol (20 μM) or N-Acetyl Cystein (N-NAC100 μM). Data are mean±SEM of 4 independent experiments. FIG. 4j shows representative confocal images acquired from INS-1 cells transfected with mitochondrial VDAC1 (mtVDAC1) or plVDAC1 and cultured with either 5 mM glucose (5G) or 20 mM glucose (20G) for 24 h. Green (Calcein) and red (Ethidium homodimer-1, EthD1) indicate live and dead cells respectively. FIG. 4k: average of ratios calculated by division of EthD1 intensity to calcein intensity under the same conditions as in (j). Data are mean±SEM from 3 independent experiments. **p<0.01. FIGS. 4l, 4m: Total VDAC1 expression in the presence or absence of VDAC1ab (10 nM), metformin (20 μM) (Met), AKOS022075291 (20 μM) (AKOS) or VBIT-4 (20 μM) in INS-1 cells cultured for 72 h at either 5 mM (FIG. 4l) or 20 mM glucose (FIG. 4m). Results are mean±SEM of 3 independent experiments with four technical replicates. *p<0.05

FIGS. 5a-5f. Comparison of mtVDAC1 and desCys127/232 VDAC (Des-CysVDAC1) effects in INS-1 cell function. FIG. 5a: Surface density of mtVDAC1 and desCys(127/232)VDAC1 in INS-1 cells; Mean±SEM of 30 cells each from 3 independent experiments. FIG. 5b: Cytosolic ATP/ADP ratio measured in single INS-1 cells (Ex/Em 488/520, 37° C.) by confocal microscopy after co-transfection with PercevalHRand either mtVDAC1 or desCys(127/232)VDAC1. FIG. 5c. Glucose-induced increases in cytosolic ATP/ADP ratio are largely preserved in desCys (127/232)VDAC1 and abolished in mtVDAC1 overexpressing INS-1 cells. Area under curve (AUC) for glucose stimulation of 5-10 analyzed cells from 6 experiments and for AUC of the values after oligomycin addition. FIGS. 5d, 5e, 5f show ATP content (d), ATP release (e) and insulin release (f) from INS-1 cells transfected with empty plasmid (EP), DesCys(127/232)VDAC1 or mtVDAC1 plasmids and incubated at 1 mM (1G) or 16.7 mM glucose (16.7G) for 1 h. Bar indicates 50 um *p<0.05, **p<0.01, ***p<0.001.

FIGS. 6a-6h show Inhibition of mistargeted VDAC1 restores GSIS in pre-diabetic mice and human T2D islets. One h exposure to VDAC1 antibody (10 nM) or metformin (20 μM) restores impaired glucose-stimulated ATP generation in islets (FIG. 6a) of diabetic db/db mice in parallel with suppression of ATP release (FIG. 6b) and augments GSIS (FIG. 6c). Mean±SEM (4 independent experiments). FIG. 6d: Insulin secretion in cultured ND islets at 5 or 20 mM glucose (72 h) in the presence and absence of VDAC1 antibody or metformin, followed by 1 h incubation at 1G or 16.7G. FIG. 6e, Acute addition of VDAC1 inhibitors (1 h) improves glucose-stimulated ATP generation in islets from T2D donors. FIG. 6f: Improved GSIS in the T2D islets shown in (e). Mean±SEM (3-6 donors). FIGS. 6g, 6h, show the effect of VDAC1 antibody (10 nM) (VDAC1ab) or metformin (20 μM) (Met) on reductive capacity (tetrazolium salt reduction) in islets from non-diabetic (FIG. 6g) and T2D (FIG. 6h) organ donors. Data are mean±SEM of 7-9 non-diabetic and 3-5 T2D donors.

FIGS. 7a-7l demonstrate aberrant localization of Vdac1 and effects of its blockade in db/db mouse islets. FIG. 7a: representative confocal images showed that Vdac1 expressed predominantly on the surface of β-cells in db/db mouse islets. FIG. 7b: surface and cytosolic mean intensity of Vdac1 in β-cells were measured. FIG. 7c: VDAC1 mRNA measured by qPCR in C57/BL and db/db mouse islets. FIG. 7d, 7e: VDAC1 antibody (10 nM), metformin, AKOS022075291 and VBIT-4 (all at 20 μM) do not affect ATP content (FIG. 7d) or GSIS (FIG. 7e) in C57/BL mice. Islets isolated from four C57/BL mice were incubated separately in a single experiment. Data are mean±SEM. *P *P0.0<0.001. FIG. 7f: The VDAC1 inhibitor VBIT-4 prevents hyperglycemia in prediabetic db/db mice injected 5 weeks (25 mg/kg daily ip) compared to vehicle treated db/db mice (n=12). C57/BL mice receiving either VBIT-4 (n=5) or vehicle (n=6) are also depicted. Six db/db mice from each group were followed for another 3-4 weeks for reversibility of the treatment. All c57/bl mice were monitored throughout. FIG. 7g, Plasma glucose concentrations during i.p. glucose tolerance test (IPGTT, 2 g/kg) in db/db or C57/BL mice after VBIT-4 treatment as in (f). Mean±SEM of 12 mice (12 db/db and 5-6 C57/BL in each group). FIG. 7h, 7i: Area under curve (AUC) for plasma glucose (7 h) and for plasma insulin (7i). *p<0.05, **p<0.01, ***p<0.001. FIG. 7j: Body weight of db/db and C57/BL mice treated with VBIT-4 (25 mg/kg ip) or vehicle for 5 weeks. FIG. 7k plasma insulin during IPGTT (2 g/kg) of the experiments shown in FIG. 7g. Mean±SEM 12 db/db in each group and 5-6 C57/BL in each group is shown. FIG. 7l: Glucose-stimulated insulin secretion from isolated islets of db/db mice treated for 5 weeks with daily IP injections of VBIT-4 (25 mg/kg) or vehicle. The islets were incubated for 1 h at 1 (1G) or 16.7 mM glucose (16.7G). Data are mean±SEM of 6 mice in each group with 2 technical replicates.

FIGS. 8a-8e shows the effect of orally administered VBIT-4 on blood glucose in model diabetic mice. VBIT-4 at the indicated doses or vehicle was administered by gavage to db/db mice daily for 5 weeks. Blood glucose concentration was measured once a week. FIG. 9 shows the effect of racemic mixture of VBIT-4 (BGD-4) and its enantiomers (BGD-4-1 and BGD-4-2) on cellular ATP release (FIG. 9a) and content (FIG. 9b) in INS-1 cells transiently transfected with a plasmid encoding plasma membrane targeted VDAC1 (plVDAC1).

FIG. 10 shows the binding of VBIT-4 and metformin to purified VDAC1. Purified VDAC1, labeled using the NanoTemper fluorescent protein-labeling Kit BLUE, was incubated with increasing concentrations of VBIT4 (0.625-100 mM) or metformin (1 to 100 mM).

### DETAILED DESCRIPTION OF THE INVENTION

The present invention relates to the use of molecules capable of specifically binding to VDAC1 and acting as inhibitors of, inter alia, its channel conductance, for treating diabetes including prevention of the development of pre-diabetic conditions to diabetes and treatments of subjects diagnosed to have diabetes. According to exemplary embodiments piperazine and/or piperidine derivatives specifically interacting with VDAC1 and acting as inhibitors of, inter alia, its channel conductance, for treating diabetes including prevention of the development of pre-diabetic conditions to diabetes and treatments of subjects diagnosed to have diabetes. Particularly exemplified derivatives are molecules designated herein VBIT-4 and AKOS. Furthermore, the present invention shows that administration of these molecules to diabetic (db/db) mice prevented the development of hyperglycemia and preserved the glucose-stimulated insulin secretion in the treated mice.

Without wishing to be bound by any specific theory or mechanism of action, the present invention discloses for the first time that β-cells of pancreatic islets obtained from T2D patients overexpress VDAC1, and such overexpression leads to the targeting of VDAC1 into the plasma membrane, where VDAC1 activity leads to loss of cellular ATP. As glucose stimulated insulin secretion (GSIS) is dependent on ATP, reduction in its level results in reduced insulin secretion and hyperglycemia. Thus, specific inhibition of VDAC1 conductance activity, particularly of VDAC1 located within the plasma membrane of diabetic pancreatic β-cells, is capable of restoring insulin secretion and normoglycemia. Accordingly, the present invention provides additional compounds, including small organic molecules, peptides and antibodies capable of specifically binding to and inhibiting VDAC1 for treating diabetes and associated conditions.

### Definitions

The term “VDAC” as used herein refers to Voltage-Dependent Anion Channel protein of a highly conserved family of mitochondrial porins. Three VDAC isoforms, VDAC Type 1 (VDAC1), VDAC Type 2 (VDAC2) and VDAC Type 3 (VDAC3), encoded by three genes, are known to date. According to certain embodiments, the term “VDAC1” as used herein refers to human VDAC1 comprising 283 amino acids (NP_003365).

The term “treating” as used herein refers to inhibiting the disease state, i.e., arresting the development of the disease state or its clinical symptoms, or relieving the disease state, i.e., causing temporary or permanent regression of the disease state or its clinical symptoms. The term is interchangeable with any one or more of the following: abrogating, ameliorating, inhibiting, attenuating, blocking, suppressing, reducing, halting, alleviating or preventing symptoms associated with the disease. According to certain exemplary embodiments, the term “treating” refers to any one of preventing the progression of prediabetic to diabetic, reducing hyperglycemia, restoring insulin secretion, preventing β-cell dysfunction, improving glucose tolerance and improving glucose-stimulated insulin secretion.

The term “inhibition” or “inhibiting” with regard to VDAC1 refers to decreasing VDCA1 channel conductance. According to certain embodiments, the reduction in channel conductance results in a decrease of ATP transport through the channel, such that ATP amount in a cell cytosol is increased upon administering of a molecule of the present invention compared to its amount in a corresponding cell without addition of the molecule.

The term “binding” as used herein and in the claims refers to binding of a molecule of the invention to VDAC1 measured by microscale thermophoresis as described in the Example section hereinafter. Alternatively, the term “binding” refers to affinity, the affinity being less than 10 μM.

The term “therapeutically effective amount” as used herein with regard to a compound of the invention is an amount of a compound that, when administered to a subject will have the intended therapeutic effect, e.g. reducing hyperglycemia, restoring insulin secretion, improving glucose tolerance or improving glucose-stimulated insulin secretion. The full therapeutic effect does not necessarily occur by administering of one dose, and may occur only after administering of a series of doses. Thus, a therapeutically effective amount may be administered in one or more doses. The precise effective amount needed for a subject will depend upon, for example, the subject's weight, health and age, the nature and severity of the diabetic condition, and optionally on the combination of the compounds of the invention with additional therapeutics, and the mode of administration.

According to one aspect, the present invention provides a substituted piperazine- and piperidine-derivative of general Formula (I) for use in treating and/or preventing the progress of diabetes in a subject in need thereof, wherein formula (I) is:

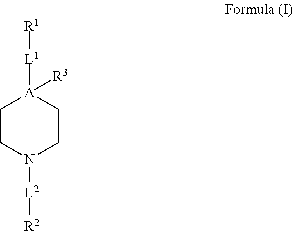

wherein:
A is carbon (C) or nitrogen (N);
R³ is absent, a hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen; wherein when A is nitrogen (N), R³ is absent;
L¹ is absent or is an amino linking group —NR⁴—, wherein R⁴ is hydrogen, a C₁₋₅-alkyl, a C₁₋₅-alkylene or a substituted alkyl —CH₂R, wherein R is a functional group selected from the group consisting of hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl; preferably R⁴ is hydrogen;
R¹ is an aromatic moiety, preferably phenyl, which may be substituted with one or more of Z;
Z is independently at each occurrence a functional group selected from the group consisting of, hydrogen, halo, haloalkyl, haloalkoxy, perhaloalkoxy or C₁₋₂-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl; preferably Z is C₁₋₂-perfluoroalkoxy; preferably R¹ is a phenyl and Z is trifluoromethoxy; preferably R¹ is a phenyl substituted with one trifluoromethoxy, most preferably at the para position;
L² is a linking group, such that when A is nitrogen (N), L² is a group consisting of 4-10 atoms (apart from hydrogen atoms), optionally forming a ring, whereof at least one of the atoms is nitrogen, said nitrogen forming part of an amide group; preferably said linking group is selected from the group consisting of an C₄₋₆-alkylamidylene and a pyrrolidinylene, said linking group optionally substituted with one or two of alkyl, hydroxy, oxo or thioxo group; most preferably L² is selected from butanamidylene, N-methylbutanamidylene, N,N-dimethylbutanamidylene, 4-hydroxybutanamidylene (HO—CH₂—C*H—CH₂—C(O)NH—, wherein the asterisk denotes attachment point), 4-oxobutanamidylene, 4-hydroxy-N-methylbutanamidylene, 4-oxo-N-methylbutanamidylene, 2-pyrrolidonyl, pyrrolidine-2,5-dionylene, 5-thioxo-2-pvrrolidinonylene and 5-methoxy-2-pvrrolidinonylene;
and when A is carbon (C), then L² is either as defined for L² when A is nitrogen (N) or C₁₋₄ alkylene; L² is preferably methylene (—CH₂—);
R² is a phenyl or a naphthyl, optionally substituted with halogen, preferably when R² is a phenyl it is substituted with halogen, preferably chlorine, at the para position, preferably when R² is naphthyl, L² is an alkylene group, preferably —CH₂—;
with a proviso that when A is carbon (C), L¹ is —NR⁴—, R⁴ is hydrogen, and R² is phenyl substituted with chlorine, then L² is not pyrrolidine-2,5-dione
or an enantiomer, diastereomer, mixture or salt thereof.

In some embodiments, R³ is hydrogen or heteroalkyl group comprising 3-12 atoms apart from hydrogen atoms, wherein at least one of said 3-12 atoms is a heteroatom, selected from nitrogen, sulfur and oxygen. In other embodiments (i.e., when A is nitrogen), R³ is absent.

In some embodiments, R⁴ is hydrogen.

In some embodiments, R¹ is a phenyl substituted trifluoromethoxy. In some embodiments, R¹ is a phenyl substituted with one trifluoromethoxy. In some embodiments, R¹ is a phenyl substituted with one trifluoromethoxy at the para position. In some embodiments, R¹ is phenyl

In some embodiments, L² is a linking group, consisting of 4-10 atoms (apart from hydrogen atoms), optionally forming a ring, whereof at least one of the atoms is nitrogen, said nitrogen forming part of an amide group; preferably said linking group is selected from the group consisting of an C₄₋₆-alkylamidylene and a pyrrolidinylene, said linking group optionally substituted with one or two of alkyl, hydroxy, oxo or thioxo group; most preferably L² is selected from butanamidylene, N-methylbutanamidylene, N,N-dimethylbutanamidylene, 4-hydroxybutanamidylene (HO—CH₂—C*H—CH₂—C(O)NH— wherein the asterisk denotes attachment point), 4-oxobutanamidylene, 4-hydroxy-N-methylbutanamidylene, 4-oxo-N-methylbutanamidylene, 2-pyrrolidonyl, pyrrolidine-2,5-dionylene, 5-thioxo-2-pyrrolidinonylene and 5-methoxy-2-pyrrolidinonylene; or L² is C₁₋₄ alkylene, preferably methylene (—CH₂—);

The term “pyrrolidinylene” refers to a pyrrolidine ring as a bivalent substituent. Pyrrolidinylene include unsubstituted and substituted rings, such as, but not limited to, pyrrolidine-2-5-dione, 2-pyrrolidinone, 5-thioxo-2-pyrrolidinone, 5-methoxy-2-pyrrolidinone and the like.

In one embodiment, when A is nitrogen (N), the linking group L² is selected from the group consisting of an C₄₋₆-alkylamidylene and a pyrrolidinylene, said linking group optionally substituted with one or two of alkyl, hydroxy, oxo or thioxo group. For example, L² may be butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methylbutanamidylene, 2-pyrrolidonyle, pyrrolidine-2,5-dionylene, 5-thioxo-2-pyrrolidinonylene or 5-methoxy-2-pyrrolidinonylene. Preferably, when L² is butanamidylene, N-methylbutanamidylene, N,N-dimethylbutanamidylene, 4-hydroxybutanamidylene, 4-oxobutanamidylene, 4-hydroxy-N-methylbutanamidylene or 4-oxo-N-methylbutanamidylene, then preferably the carbon in third position (C) of the butanamide moiety is bonded to the nitrogen (N) of the piperazine ring or the piperidine ring and the nitrogen (N) of the butanamide moiety is bonded to R². For example, when L² is 2-pyrrolidone, pyrrolidine-2,5-dione, 5-thioxo-2-pyrrolidone or 5-methoxy-2-pyrrolidone, then preferably a carbon (C) of the pyrrolidine moiety is bonded to the nitrogen (N) of the piperazine ring or the piperidine ring and the nitrogen (N) of the pyrrolidine moiety is bonded to R².

In another embodiment, A is carbon (C), R³ is heteroalkyl and L² is methylene.

The invention also relates to the stereoisomers, enantiomers, mixtures thereof, and salts, particularly the physiologically acceptable salts, of the compounds of general Formula (I) according to the invention.

According to certain embodiments, the at least one substituted piperazine- and piperidine-derivative is of general Formula Ia:

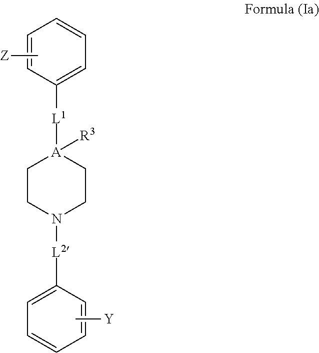

wherein:

A, R³, Z and L¹ are as previously defined in reference to compound of Formula (I); preferably A is nitrogen (N);

L^{2′} is a linking group selected from the group consisting of an C₄-alkylamidylene, C₅-alkylamidylene and C₆-alkylamidylene, optionally substituted with one or two of alkyl, hydroxy, oxo or thioxo group; preferably L^{2′} is selected from butanamidylene, N-methylbutanamidylene, N,N-dimethylbutanamidylene, 4-hydroxybutanamidylene, 4-oxobutanamidylene, 4-hydroxy-N-methylbutanamidylene or 4-oxo-N-methylbutanamidylene; most preferably L^{2′} is 4-hydroxybutanamidylene; wherein preferably the carbon (C) at position 3 of the alkyl moiety of alkylamidylene L^{2′} is bonded to the nitrogen (N) of the piperazine ring or of the piperidine ring, and the nitrogen (N) of the butanamide moiety is bonded to the phenyl group; preferably L² is HO—CH₂—C*H—CH—C(O)NH—, wherein the asterisk denotes attachment point;

Y is halogen, preferably chlorine, e.g. at the para position;

or an enantiomer, diastereomer, mixture or salt thereof.

According to certain embodiments, the substituted piperazine- and piperidine-derivative is of general Formula (Ib):

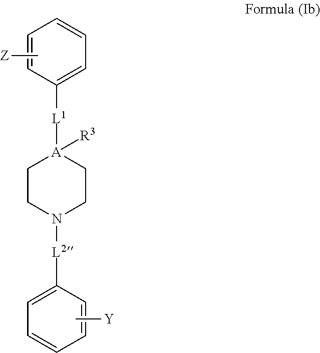

wherein:

A, R³, and Z are as previously defined in reference to the compound of Formula (I); preferably A is nitrogen (N);

L¹ is absent;

L² is a pyrrolidinylene linking group, optionally substituted with one or two of alkyl, hydroxy, oxo or thioxo group, preferably L^{2″} is selected from 2-pyrrolidonylene, pyrrolidine-2,5-dionylene, 5-thioxo-2-pyrrolidinonylene and 5-methoxy-2-pyrrolidinonylene; most preferably L^{2″} is pyrrolidine-2,5-dionylene; wherein preferably a carbon (C) at position 4 or the carbon (C) at position 3 of the pyrrolidinyl moiety L^{2″} is bonded to the nitrogen (N) of the piperazine ring or the piperidine ring and the nitrogen (N) of the pyrrolidinyl moiety is bonded to the phenyl group substituted with Y; and

Y is halogen, preferably chlorine, e.g. at the para position.

According to certain embodiments, the substituted piperazine- and piperidine-derivative is of general Formula (Ic):

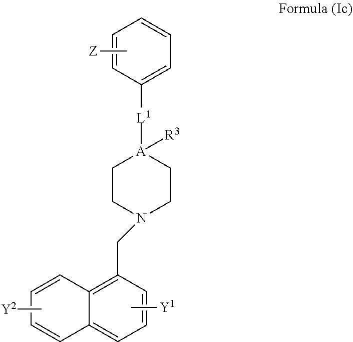

wherein:

A, R³, and Z are as previously defined in reference to the compounds of general Formula (I);

L is —NH—; and

Y¹ and Y² are each independently absent or a halogen;

or an enantiomer, diastereomer, mixture or salt thereof.

Preferred compounds of Formula (Ic) are those wherein R³ is —C(O)NHCH₂C(O)OH group, and/or wherein Z is C₁₋₂-alkoxy or halogenated C₁₋₂-alkoxy, e.g. C₁₋₂-perfluoroalkoxy.

According to certain embodiments, the substituted piperazine- and piperidine-derivatives is of general Formula (Id):

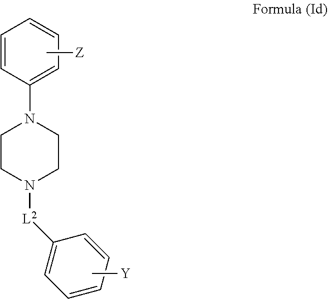

wherein

L² is selected from the group consisting of an C₄₋₆-alkylamidylene (e.g. HO—CH₂—C*H—CH₂—C(O)NH—, wherein the asterisk denotes attachment point), and a pyrrolidinylene (e.g. pyrrolidin-2,5-dionylene), optionally substituted with one or two of alkyl, hydroxy, oxo or thioxo group; and

Z is haloalkoxy, e.g. C₁₋₂-perfluoroalkoxy, and Y is halogen.

The invention also relates to the stereoisomers, enantiomers, mixtures thereof and salts thereof, of the compounds of general Formulae (Ia), (Ib), (Ic), and (Id), according to the invention.

Table 1 provides non-limiting examples of compound of general Formula (I). It includes compounds as follows:
N-(4-chlorophenyl)-4-hydroxy-3-(4-(4-(trifluoromethoxy)phenyl)-piperazin-1-yl)butanamide (Formula 1);
1-(4-chlorophenyl)-3-(4-(4-(trifluoromethoxy)phenyl)piperazin-1-yl)pyrrolidine-2,5-dione (Formula 2);
1-(naphthalen-1-yl)methyl)-4-(phenylamino)-piperidine-4-carbonyl)glycine (Formula 3);
1-(4-chlorophenyl)-3-(4-(4-(trifluoromethoxy)phenyl)piperazin-1-yl)pyrrolidin-2-one (Formula 4);
1-(4-chlorophenyl)-5-thioxo-3-(4-(4-(trifluoro-methoxy)phenyl)piperazin-1-yl)pyrolidin-2-one (Formula 5);
1-(4-chlorophenyl)-5-methoxy-4-(4-(4-(trifluoromethoxy)phenyl)-piperazin-1-yl)pyrrolidin-2-one (Formula 6);
1-(4-chlorophenyl)-5-thioxo-4-(4-((4-(trifluoromethoxy)phenyl)amino)piperidin-1-yl)pyrrolidin-2-one (Formula 7);
4-(4-chlorophenyl)-4-oxo-3-(4-(4-(trifluoromethoxy)phenyl)piperazin-1-yl)butanamide (Formula 8); and
N-(4-chlorophenyl)-4-hydroxy-N-methyl-3-(4-(4-(trifluoro-methoxy)phenyl)piperazin-1-yl)butanamide (Formula 9).

TABLE 1
examples of compound of general Formula (I)

| Formula | | Description |
|---|---|---|
| # | Structure | (according to general Formula (I)) |
| 1 | 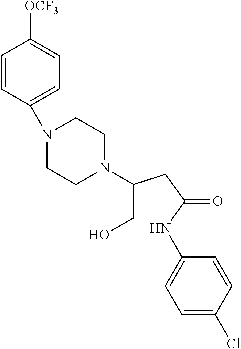 [SMILES: O=C(CC(CO)N1CCN(c2ccc(OC(F)(F)F)cc2)CC1)Nc1ccc(Cl)cc1] | A is nitrogen (N), R³ is absent, L¹ is absent, R¹ is phenyl substituted with one trifluoromethoxy, L² is 4- hydroxybutanamidylene, the 3^{rd} carbon (C) of the butanamide moiety is bonded to the nitrogen (N) of the piperazine ring, the nitrogen (N) of the butanamide moiety is bonded to R² and R² is a phenyl substituted with chlorine at the para position [also identified herein as VBIT-4 or as BGD-4] |
| 2 | 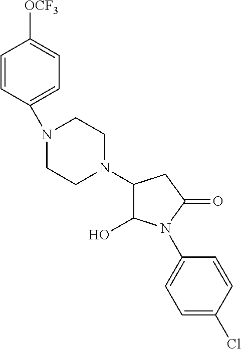 [SMILES: O=C1CC(N2CCN(c3ccc(OC(F)(F)F)cc3)CC2)C(O)N1c1ccc(Cl)cc1] | A is nitrogen (N), R³ is absent, L¹ is absent, R¹ is phenyl substituted with one trifluoromethoxy, L² is pyrrolidine-2,5-dione, the carbon (C) at position 3 of the pyrrolidine moiety is bonded to the nitrogen (N) of the piperazine ring, the nitrogen (N) of the pyrrolidine moiety is bonded to R² and R² is a phenyl substituted with chlorine at the para position [also identtified herein as VBIT-3 or as BGD-3] |
| 3 | 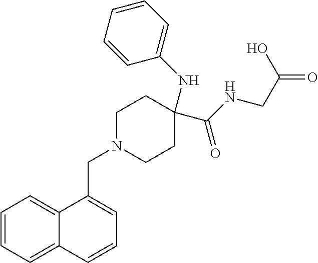 [SMILES: O=C(O)CNC(=O)C1(Nc2ccccc2)CCN(Cc2cccc3ccccc23)CC1] | A is carbon (C), R³ is —C(O)NHCH₂C(O)OH group; L¹ is —NH—, R¹ is a phenyl, L² is methylene and R² is a naphthyl [also identified herein as VBIT-12] |
| 4 | 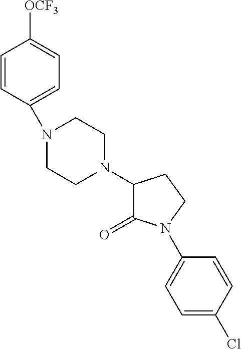 [SMILES: O=C1C(N2CCN(c3ccc(OC(F)(F)F)cc3)CC2)CCN1c1ccc(Cl)cc1] | A is nitrogen (N), R³ is absent, L¹ is absent, R¹ is a phenyl substituted with one trifluoromethoxy; L² is 2- pyrrolidone, the carbon (C) at position 3 of the pyrrolidone moiety is bonded to the nitrogen (N) of the piperazine ring, the nitrogen (N) of the pyrrolidone moiety is bonded to R² and R² is a phenyl substituted with chlorine at the para position [also identified herein as VBIT-5] |
| 5 | 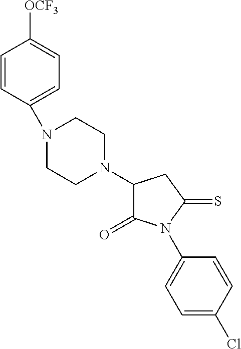 [SMILES: O=C1C(N2CCN(c3ccc(OC(F)(F)F)cc3)CC2)CC(=S)N1c1ccc(Cl)cc1] | A is nitrogen (N), R³ is absent, L¹ is absent, R¹ is a phenyl substituted with one trifluoromethoxy, L² is 5- thioxo-2-pyrrolidone, the carbon (C) at position 3 of the pyrrolidine moiety is bonded to the nitrogen (N) of the piperazine ring, the nitrogen (N) of the pyrrolidine moiety is bonded to R² and R² is a phenyl substituted with chlorine at the para position [also identified herein as VBIT-6] |
| 6 | 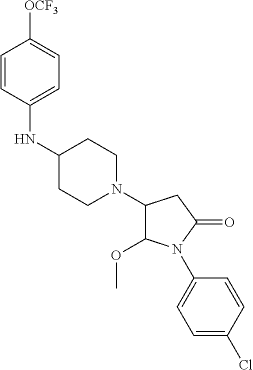 [SMILES: COC1C(N2CCC(Nc3ccc(OC(F)(F)F)cc3)CC2)CC(=O)N1c1ccc(Cl)cc1] | A is carbon (C), R³ is hydrogen, L¹ is —NH—, R¹ is a phenyl substituted with one trifluoromethoxy, L² is 5- methoxy-2-pyrrolidinone, the carbon (C) at position 4 of the pyrrolidine moiety is bonded to the nitrogen (N) of the piperidine ring, the nitrogen (N) of the pyrrolidine moiety is bonded to R² and R² is a phenyl substituted with chlorine at the para position [also identified herein as VBIT-9] |
| 7 | 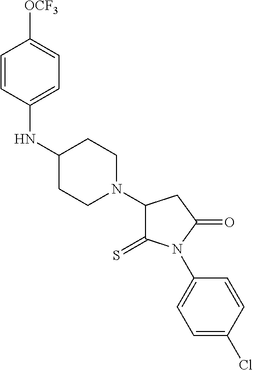 [SMILES: O=C1CC(N2CCC(Nc3ccc(OC(F)(F)F)cc3)CC2)C(=S)N1c1ccc(Cl)cc1] | A is carbon (C), R³ is hydrogen, L¹ is —NH—, R¹ is a phenyl substituted with one trifluoromethoxy, L² is 5- thioxo-2-pyrrolidone, the carbon (C) at position 3 of the pyrrolidine moiety is bonded to the nitrogen (N) of the piperidine ring, the nitrogen (N) of the pyrrolidine moiety is bonded to R² and R² is a phenyl substituted with chlorine at the para position [also identified herein as VBIT-10] |
| 8 | 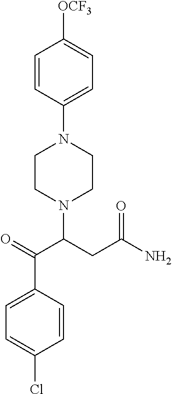 [SMILES: NC(=O)CC(C(=O)c1ccc(Cl)cc1)N1CCN(c2ccc(OC(F)(F)F)cc2)CC1] | A is nitrogen (N), R³ is absent, L¹ is absent, R¹ is phenyl substituted with one trifluoromethoxy, L² is 4- oxobutanamide, the 3^{rd} carbon (C) of the butanamide moiety is bonded to the nitrogen (N) of the piperazine ring, the 4^{th} carbon (C) of the butanamide moiety is bonded to R² and R² is a phenyl substituted with chlorine at the para position [also identified herein as VBIT-7] |
| 9 | 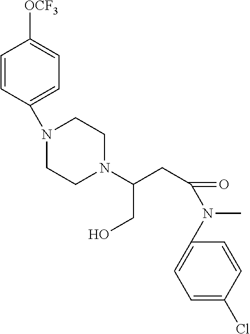 [SMILES: CN(C(=O)CC(CO)N1CCN(c2ccc(OC(F)(F)F)cc2)CC1)c1ccc(Cl)cc1] | A is nitrogen (N), R³ is absent, L¹ is absent, R¹ is phenyl substituted with one trifluoromethoxy, L² is 4- hydroxy-N-methylbutanamide, the 3^{rd} carbon (C) of the butanamide moiety is bonded to the nitrogen (N) of the piperazine ring, the nitrogen (N) of the butanamide moiety is bonded to R² and R² is a phenyl substituted with chlorine at the para position [also identified herein as VBIT-8] |

Some terms used herein to describe the compounds according to the invention are defined more specifically below.

The term “hydroxyl” as used herein refers to an OH group.

The term “halogen” as used herein denotes an atom selected from among F, Cl, Br and I, typically Cl and Br.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like.

The term “heteroalkyl” as used herein in reference to R³ moiety of the general Formulae (I), (Ia), (Ib), (Ic), (Id), (II) and (IIa), refers to a saturated or unsaturated group of 3-12 atoms (apart from hydrogen atoms), wherein one or more (typically 1, 2 or 3) atoms are a nitrogen, oxygen, or sulfur atom, for example an alkyloxy group, as for example methoxy or ethoxy, or a methoxymethyl-, nitrile-, methylcarboxyalkylester- or 2,3-dioxyethyl-group; typically heteroalkyl group is a chain comprising an alkylene, and at least one of a carboxylic acid moiety, a carbonyl moiety, an amine moiety, a hydroxyl moiety, an ester moiety, an amide moiety. The term heteroalkyl refers furthermore to a carboxylic acid or a group derived from a carboxylic acid as for example acyl, acyloxy, carboxyalkyl, carboxyalkylester, such as for example methylcarboxyalkylester, carboxyalkylamide, alkoxycarbonyl or alkoxycarbonyloxy; typically the term refers to —C(O)NHCH₂C(O)OH group.

The term “C₁-n-alkyl”, wherein n may have a value as defined herein, denotes a saturated, branched or unbranched hydrocarbon group with 1 to n carbon (C) atoms. Examples of such groups include methyl, ethyl, n-propyl, iso-propyl, butyl, iso-butyl, sec-butyl, tert-butyl, n-pentyl, iso-pentyl, neo-pentyl, tert-pentyl, n-hexyl, iso-hexyl, etc.

The term C₁₋₄-alkyl denotes a saturated, branched or unbranched hydrocarbon group with 1 to 4 carbon (C) atoms.

The term “C₁-n-alkoxy”, wherein n may have a value as defined herein, denotes an alkyl group as defined herein, bonded via —O— (oxygen) linker. The term “C₁₋ₙ alkylene”, wherein n may have a value as defined herein, denotes an alkylene group of saturated hydrocarbons substituents with the general formula CₙH₂ₙ. Generally, n is a positive integer. For example, C₁ alkylene refers to methylene (—CH₂—), C₃ alkylene refers to C₃H₆, which may be n-propylene (—CH₂CH₂CH₂—) or isopropylene (—CH(CH₃)CH₂— or —CH₂CH(CH₃)—). Preferably the term refers to an unbranched n-alkylene.

The term “C₁₋ₙ-perfluoroalkoxy”, wherein n may have a value as defined herein, denotes an alkoxy group with hydrogen atoms substituted by fluorine atoms.

The term “C₁₋ₘ-alkylamidyl”, wherein m may have a value as defined herein, denotes a group comprising 1 to m carbon (C) atoms and an amide group formed by either Cₘ₋ₐalkyl-COOH and H₂N—Cₐalkyl, or Cₘ₋ₐalkyl-NH₂ and HOOC—Cₐalkyl, wherein a is smaller than or equal to m. Similarly, the terms C₄-alkylamidylene, C₅-alkylamidylene and C₆-alkylamidylene refer to divalent Cₘ-alkylamidyl groups, wherein m is either 4, 5, or 6, respectively.

The invention also relates to the stereoisomers, such as diastereomers and enantiomers, mixtures and salts, particularly the physiologically acceptable salts, of the compounds of general Formulae (I), (Ia), (Ib), (Ic), and (Id), and of the compounds of structural formulae 1, 2, 3, 4, 5, 6, 7, 8 and 9.

The compounds of the present invention can have asymmetric centers at any of the atoms. Consequently, the compounds can exist in enantiomeric or diastereomeric forms or in mixtures thereof.

The compounds of general Formulae (I), (Ia), (Ib), (Ic), and (Id), or intermediate products in the synthesis of compounds of general Formulae (I), (Ia), (Ib), (Ic), and (Id), may be resolved into their enantiomers and/or diastereomers on the basis of their physical-chemical differences using methods known in the art. For example, cis/trans mixtures may be resolved into their cis and trans isomers by chromatography. For example, enantiomers may be separated by chromatography on chiral phases or by recrystallization from an optically active solvent or by enantiomer-enriched seeding.

The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. The present invention intends to encompass all structural and geometrical isomers including cis, trans, E and Z isomers.

According to certain exemplary embodiments, the present invention provides a racemic mixture of a compound having structural formula 1 (designated herein VBIT-4 or BGD-4) for use in treating and/or preventing the progression of diabetes.

According to additional certain exemplary embodiments, the present invention provides an optically pure (+) enantiomer of a compound having structural formula 1 for use in treating and/or preventing the progression of diabetes.

According to additional certain exemplary embodiments, the present invention provides an optically pure (−) enantiomer of a compound having structural formula 1 for use in treating and/or preventing the progression of diabetes.

The compounds of general Formulae (I), (I), (Ia), (Ib), (Ic), and (Id), and the compounds of structural formulae 1, 2, 3, 4, 5, 6, 7, 8 and 9, may be converted into the salts thereof, particularly physiologically acceptable salts for pharmaceutical use. The term “salt” encompasses both basic and acid addition salts, including but not limited to, carboxylate salts or salts with amine nitrogens, and include salts formed with the organic and inorganic anions and cations discussed below. Furthermore, the term includes salts that form by standard acid-base reactions with basic groups (such as amino groups) and organic or inorganic acids. Such acids include, but are not limited to, hydrochloric acid, hydrobromic acid, hydrofluoric acid, trifluoroacetic hydrobromic acid, sulfuric hydrobromic acid, phosphoric hydrobromic acid, acetic acid, succinic acid, citric acid, lactic acid, maleic, fumaric, palmitic acid, cholic acid, pamoic acid, mucic acid, D-glutamic acid, D-camphoric acid, glutaric acid, phthalic acid, tartaric acid, lauric acid, stearic acid, salicylic acid, methanesulfonic acid, benzenesulfonic acid, sorbic acid, picric acid, benzoic acid, or cinnamic acid. Each possibility represents a separate embodiment of the invention. Compounds of general Formulae (I¹), (I), (Ia), (Ib), (Ic) and (Id), containing a carboxy group, may be converted into the salts thereof, particularly into physiologically acceptable salts for pharmaceutical use, with organic or inorganic bases. Suitable bases for this purpose include, for example, sodium hydroxide, potassium hydroxide, ammonium hydroxide, arginine or ethanolamine. Each possibility represents a separate embodiment of the invention.

According to another aspect, the present invention provides a compound of general formula (IIa) for use in treating and/or preventing the progress of diabetes in a subject in need thereof, wherein formula (IIa) is:

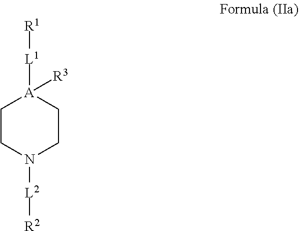

wherein:

A is carbon (C);

R³ is a hydrogen, an unsubstituted or substituted amide or a heteroalkyl group comprising 3-12 atoms apart from hydrogen atoms, wherein at least one of said 3-12 atoms is a heteroatom, selected from nitrogen, sulfur and oxygen;

L¹ is an amino linking group —NR⁴—, wherein R⁴ is hydrogen, a C₁₋₅-alkyl, a C₁₋₅-alkylene or a substituted alkyl —CH₂R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl or heteroaryl;

when R³ is hydrogen, then L¹ is preferably —NH—; when R³ is heteroalkyl group comprising 3-12 atoms, then L¹ is preferably —NCₙH₂ₙ—, such that it forms a ring with R³.

R¹ is an aromatic moiety, which is optionally substituted with one or more of C₁₋₂-alkoxy, e.g. haloalkoxy, such as C₁₋₂-perfluoroalkoxy;

L² is a linking group consisting of 4-10 atoms (apart from hydrogen atoms), optionally forming a ring, whereof at least one of the atoms is nitrogen, said nitrogen forming part of an amide group or L² is C₁₋₅ alkyl or C₁₋₅alkylene; said linking group L² bonds piperidine or piperazine moiety at nitrogen (N) atom; preferably, L² is selected from butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methylbutanamidylene, 2-pyrrolidonylene, pyrrolidine-2,5-dionylene, 5-thioxo-2-pyrrolidinonylene and 5-methoxy-2-pyrrolidinonylene; and

R² is an aryl, optionally substituted with halogen, optionally when R² is a phenyl it is substituted with halogen, further optionally when R² is naphthyl, L² is an alkylenyl group. In a specific embodiment, R³ is hydrogen, L¹ is —NH—, and R¹ is a phenyl substituted with trifluoromethoxy.

The invention also relates to use of the stereoisomers, enantiomers, mixtures thereof, and salts, particularly the physiologically acceptable salts, of the compounds of general Formula (IIa).

In some embodiments, A is carbon (C), R³ is hydrogen (H), L¹ is a NH group, R¹ is a phenyl substituted with one trifluoromethoxy, L² is pyrrolidine-2,5-dione, and R² is a phenyl substituted with a chlorine at the para position.

In some embodiments, A is carbon (C), R³ is a C(O)NCH₂C(O)OH group and is connected to both A and L¹, L¹ is a NCH₂ group and is connected to both R¹ and R³, R¹ is a phenyl, L² is methylene C¹ alkylene and R² is a naphthyl.

According to certain exemplary embodiments, the present invention provides the use of compounds according to the general Formula (IIa), having the structural Formulae 10 and 11:

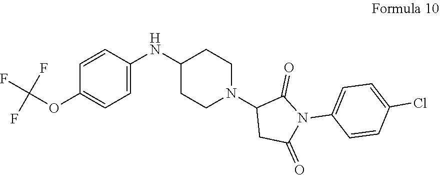

The compound of Formula 10 is also identified herein as AKOS or AKOS022075291.

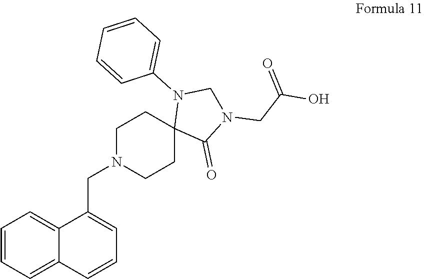 [SMILES: O=C(O)CN1CN(c2ccccc2)C2(CCN(Cc3cccc4ccccc34)CC2)C1=O]

The compound of Formula 11 is also identified herein as DIV 00781.

The compounds of general Formula (IIa) such as, without being limited to, the compounds of structural formulae 10 and 11, may be converted into the salts thereof, particularly physiologically acceptable salts for pharmaceutical use. Suitable salts of the compounds of general Formulae (IIa), such as, without being limited to, the compounds of structural formulae 10 and 11, may be formed with organic or inorganic acids, such as, without being limited to hydrochloric acid, hydrobromic acid, sulphuric acid, phosphoric acid, lactic acid, acetic acid, succinic acid, citric acid, palmitic acid or maleic acid. Each possibility represents a separate embodiment of the invention. Compounds of general Formula (IIa) containing a carboxy group, may be converted into the salts thereof, particularly into physiologically acceptable salts for pharmaceutical use, with organic or inorganic bases. Suitable bases for this purpose include, for example, sodium salts, potassium salts, arginine salts, ammonium salts, or ethanolamine salts. Each possibility represents a separate embodiment of the invention.

Prediabetes is a condition in which the fasting blood sugar level is higher than the normal or in which there is impaired tolerance to a glucose challenge (IGT, impaired glucose tolerance), but other symptoms of diabetes are missing. A subject with pre-diabetes dysregulated sugar level is highly prone to develop T2D, although changing the life style in terms of exercising and implementing suitable diet may delay or even prevent the development of diabetes.

According to certain embodiments, the use of compounds of general Formulae (I¹), (I), (Ia), (Ib), (Ic), (Id), and (IIa), particularly the specific compounds of Formulae 1 and 10 for preventing the progress of diabetes comprises preventing the progression of pre-diabetes to diabetes.

According to certain embodiments, the use of compounds of general Formulae (I¹), (I), (Ia), (Ib), (Ic), (Id), and (IIa), particularly the specific compounds of Formulae 1 and 10 for treating diabetes comprises improving glucose tolerance.

According to certain embodiments, the use of compounds of general Formulae (I¹), (I), (Ia), (Ib), (Ic), (Id), and (IIa), particularly the specific compounds of Formulae 1 and 10 for treating diabetes comprises inducing glucose-stimulated insulin secretion.

According to certain embodiments, the use of compounds of general Formulae (I¹), (I), (Ia), (Ib), (Ic), (Id), and (IIa), particularly the specific compounds of Formulae 1 and 10 for treating diabetes comprises restoring insulin secretion from pancreatic β-cells of a subject affected with diabetes.

According to certain embodiments, the use of compounds of general Formulae (I¹), (I), (Ia), (Ib), (Ic), (Id), and (IIa), particularly the specific compounds of Formulae 1 and 10 for treating diabetes comprises prevention of β-cell dysfunction. According to additional aspect, the present invention provides a method for treating diabetes and/or preventing the progress of diabetes in a subject in need thereof, the method comprises administering to the subject a therapeutically effective amount of at least one compound of general formula (I¹):

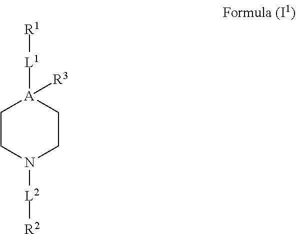

wherein:

A is carbon (C) or nitrogen (N);

R³ is absent, a hydrogen, an unsubstituted or substituted amide, or a heteroalkyl comprising 3-12 atoms (apart from hydrogen atoms), wherein at least one atom is a nitrogen, sulfur or oxygen atom, wherein when A is nitrogen (N), R³ is absent;

L¹ is absent or is an amino linking group —NR⁴—, wherein R⁴ is hydrogen, a C₁-alkyl, a C₁₋₅-alkylene or a substituted alkyl —CH₂R, wherein R is a functional group selected from the group consisting of hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl;

R¹ is an aromatic moiety, which is optionally substituted with one or more of Z;

Z is independently at each occurrence a functional group selected from the group consisting of, hydrogen, halo, haloalkyl, haloalkoxy, perhaloalkoxy or C₁₋₂-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl;

L² is a linking group, such that when A is nitrogen (N), L² is a group consisting of 4-10 atoms, apart from hydrogen atoms, optionally forming a ring, whereof at least one of the atoms is nitrogen, said nitrogen forming part of an amide group; and when A is carbon (C), then L² is selected from C₁₋₄ alkylene or a group consisting of 4-10 atoms, apart from hydrogen atoms, optionally forming a ring, whereof at least one of the atoms is nitrogen, said nitrogen forming part of an amide group;

R² is a phenyl or a naphthyl, optionally substituted with halogen;

or an enantiomer, diastereomer, mixture or salt thereof.

According to certain embodiments, the compound is selected from the group consisting of a compound of general Formulae (Ia), (Ib), (Ic) and (Id).

According to certain embodiments the compound is selected from the group consisting of a compound of structural formulae 1, 2, and 3.

According to certain embodiments the compound is of Formula (IIa):

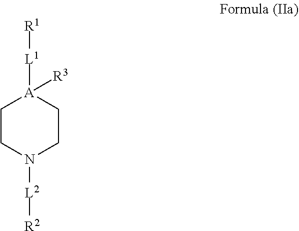

wherein:

A is carbon (C);

R³ is hydrogen or heteroalkyl chain comprising 3-12 atoms, apart from hydrogen atoms, wherein at least one is a heteroatom, selected from nitrogen, sulfur and oxygen;

L¹ is an amino linking group —NR⁴—, wherein R⁴ is hydrogen, a C₁₋₅-alkyl, a C₁₋₅-alkylene or a substituted alkyl —CH₂R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl or heteroaryl;

when R³ is heteroalkyl group comprising 3-12 atoms, apart from hydrogen atoms, then L forms a ring with R³;

R¹ is an aromatic moiety, which is optionally substituted with one or more of C₁₋₂-alkoxy, and/or C₁₋₂-perfluoroalkoxy;

L² is a linking group consisting of 4-10 atoms, apart from hydrogen atoms, optionally forming a ring, whereof at least one of the atoms is nitrogen, said nitrogen forming part of an amide group or L² is C₁₋₅ alkyl or C₁₋₅ alkylene; said linking group L² bonds piperidine or piperazine moiety at nitrogen (N) atom; and

R² is an aryl, optionally substituted with halogen, optionally when R² is a phenyl it is substituted with halogen, further optionally when R² is naphthyl, L² is an alkylenyl group;

or an enantiomer, diastereomer, mixture or salt thereof.

According to certain embodiments the compound is selected from a compound of structural formulae 10 and 11.

According to certain embodiments preventing the progress of diabetes comprises preventing the progression of pre-diabetes to diabetes.

According to certain embodiments treating diabetes comprises at least one of inducing glucose-stimulated insulin secretion; improving glucose tolerance; restoring insulin secretion from pancreatic β-cells of a subject affected with diabetes; and prevention of β-cell dysfunction.

According to certain embodiments the subject in need thereof is a human subject.

According to certain embodiments the human subject is selected from pre-pubertal child, post-pubertal child, adolescent and an adult.

As exemplified herein below, the compounds of general Formulae (I¹), (I), (Ia), (Ib), (Ic), (Id), and (IIa), particularly the specific compounds of Formulae 1 and 10, are specific inhibitors of VDAC1 expression and of cellular ATP loss in β-cells of diabetic subjects and in normal β-cells exposed to glucotoxic conditions.

Thus, according to additional aspects, the present invention provides a method of inhibiting ATP loss from diabetic pancreatic β-cells, the method comprising exposing the diabetic pancreatic β-cells to at least one compound of the present invention.

According to certain embodiments, the compound is selected from the group consisting of a compound of general formula (I), (Ia), (Ib), (Ic), (Id), and (IIa).

According to additional aspect, the present invention provides a compound selected from the group consisting of a compound of general formula (I¹), (I), (Ia), (Ib), (Ic), (Id), and (IIa) for use in inhibiting ATP loss from diabetic pancreatic β-cells.

According to additional aspect, the present invention provides a method for treating diabetes and/or preventing the progress of diabetes in a subject in need thereof, comprising administering to the subject a therapeutically effective amount of a compound specifically binding to and inhibiting VDAC1 or a pharmaceutical composition comprising same.

According to certain embodiments, the compound specifically binds to and inhibits VDAC1 expressed on pancreatic β-cells.

According to certain embodiments, the compound inhibits ATP transport via VDAC1.

According to certain embodiments, the compound is not significantly binding to and/or inhibiting VDAC2.

According to certain embodiments, the compound is selected from the group consisting of a small organic molecule, a peptide and an antibody.

According to certain exemplary embodiments, the compound is an antibody.

According to certain embodiments, preventing the progress of diabetes comprises preventing progress of prediabetes to diabetes.

According to additional aspect, the present invention provides a compound specifically binding to and inhibiting VDAC1 for use in treating diabetes and/or preventing the progress of diabetes in a subject in need thereof. According to certain embodiments, the VDAC1 is expressed on pancreatic β-cells. According to certain embodiments, the VDCA1 is expressed on the surface of the pancreatic β-cells. In a further embodiment diabetes is type I. In another embodiment diabetes is type II. In a still further embodiment diabetes is non-insulin dependent. In a further embodiment diabetes is insulin dependent.

According to certain embodiments, the compound specifically binds to and inhibits VDAC1 expressed on pancreatic β-cells.

According to certain embodiments, the compound inhibits ATP transport via the VDAC1.

According to certain embodiments, the compound does not significantly bind to and/or inhibit VDAC2.

According to certain embodiments, the compound is selected from the group consisting of a small organic molecule, a peptide and an antibody.

In a further embodiment the small organic molecule is less than 900 Da. Typically such small organic molecule comprises a 5- or 6-membered heterocycle containing at least one heteroatom selected from N, S, and O. In some embodiments the heteroatom is selected from N and O such as a piperazine and/or piperidine ring. Moreover, in a further embodiment the small organic molecule comprises a 5- or 6-membered heterocycle containing at least one heteroatom selected from N, S, and O, wherein the heterocycle is linked to an aromatic ring or a heteroaromatic ring, such as two aromatic rings, or two heteroaromatic rings or one aromatic ring and one heteroaromatic ring.

According to certain exemplary embodiments, the compound is an antibody. Typically the antibody is a monoclonal antibody, such as a recombinant antibody.

The term “antibody” is used in the broadest sense and includes monoclonal antibodies (including full length or intact monoclonal antibodies), polyclonal antibodies, multivalent antibodies, multi-specific antibodies (e.g., bi-specific antibodies), and antibody fragments long enough to exhibit the desired biological activity, that is binding to and inhibiting VDAC1.

Antibody or antibodies according to the invention include intact antibodies, such as polyclonal antibodies or monoclonal antibodies (mAbs), as well as proteolytic fragments thereof, such as the Fab or F(ab′)₂ fragments. Single chain antibodies also fall within the scope of the present invention.

According to certain embodiments, treating diabetes comprises at least one of inducing glucose-stimulated insulin secretion; improving glucose tolerance; restoring insulin secretion from pancreatic β-cells of a subject affected with diabetes or prediabetes; and prevention of β-cell dysfunction.

### Therapeutic Use

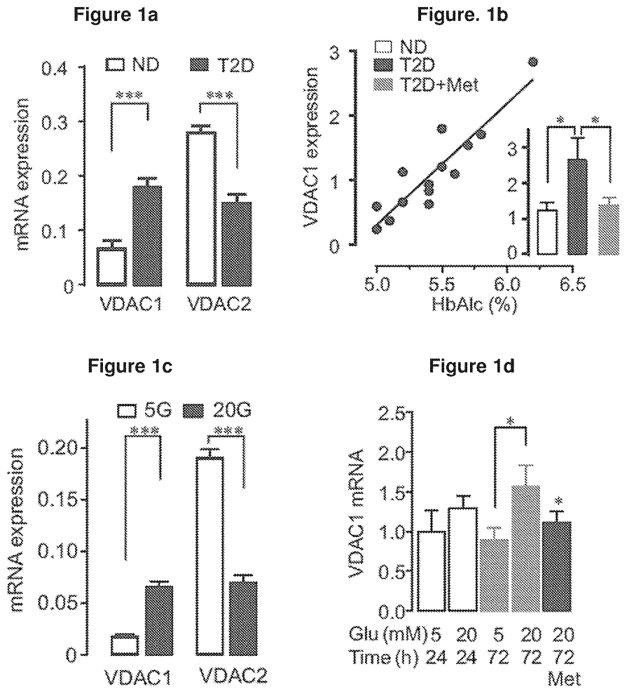

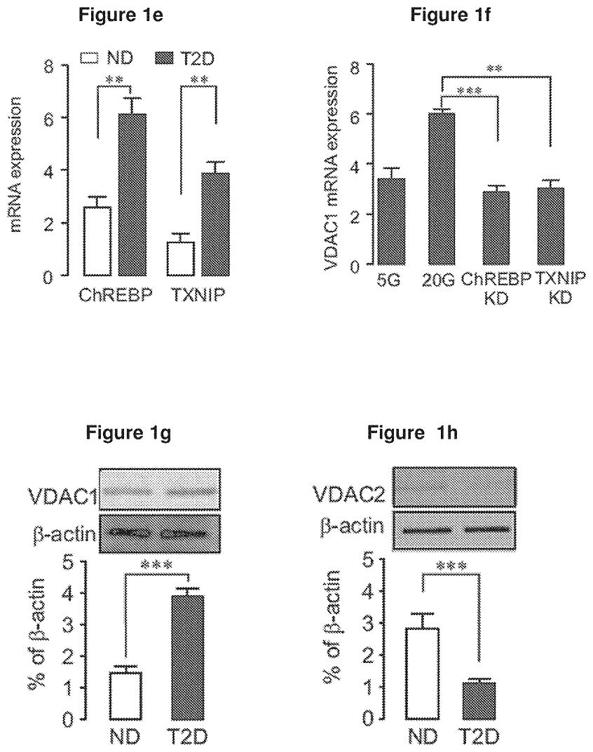

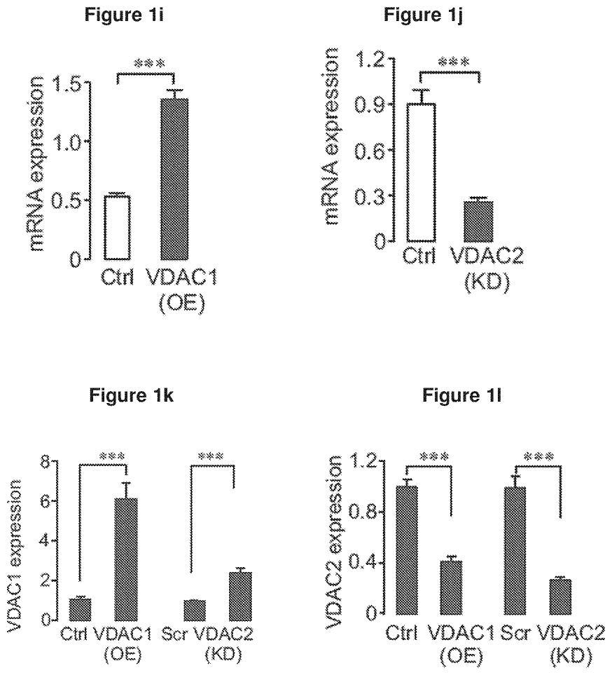

The present invention now shows that VDAC1 mRNA and protein levels were increased in pancreatic islets from T2D organ donors (donor characteristics are presented in Table 2 hereinafter), while VDAC2 was repressed (FIGS. 1a, 1g and 1h).

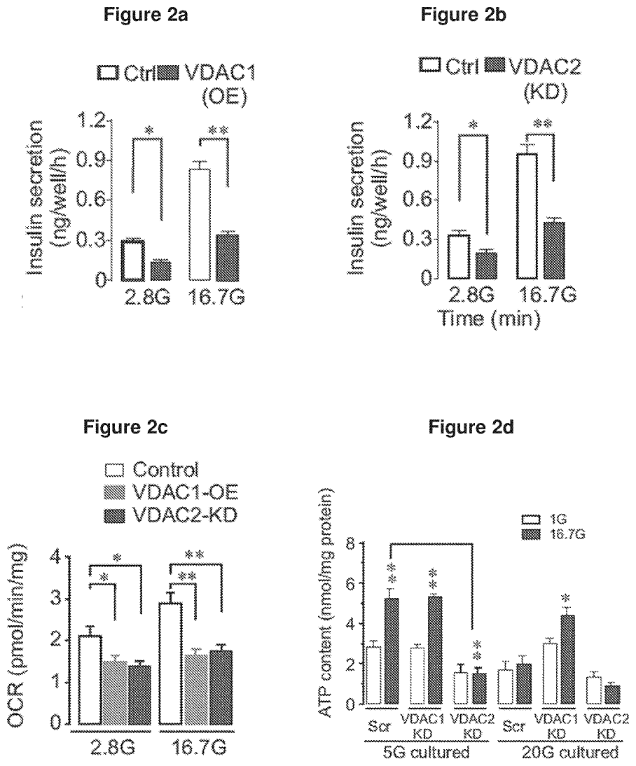

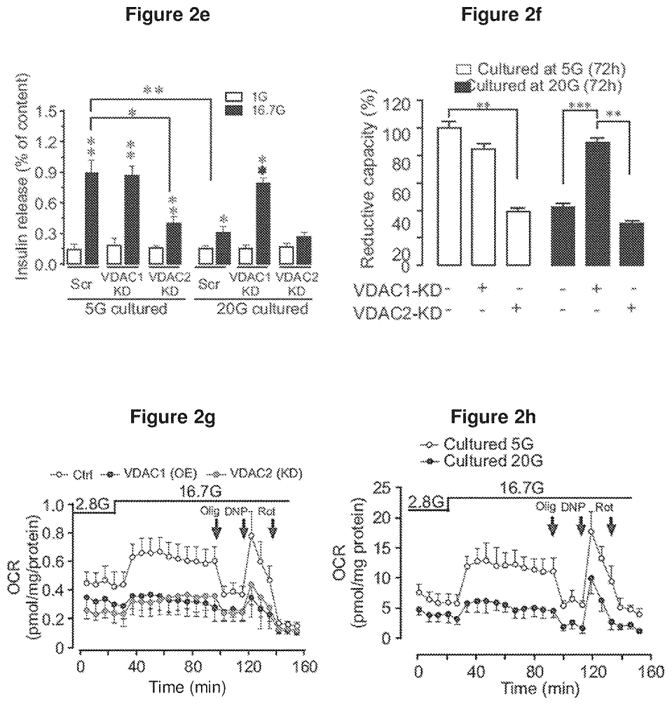

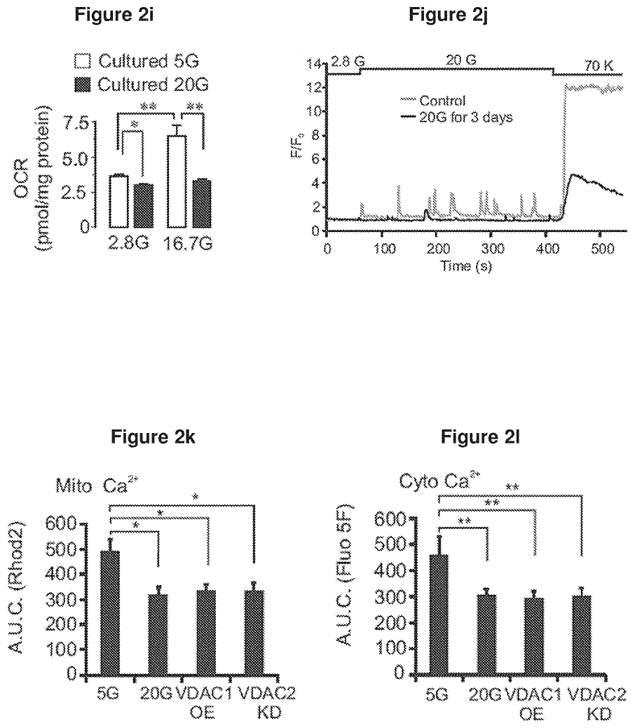

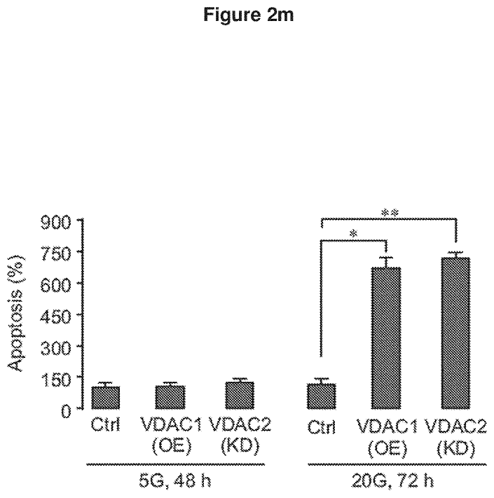

VDAC1 mRNA level was strikingly correlated with average blood glucose (glycated hemoglobinA1c, HbA1c) in islets from non-diabetic donors (FIG. 1b). Culture of human islets under glucotoxic conditions (20 mM glucose for 72 h) similarly showed increased VDAC1 and decreased VDAC2 mRNA level (FIG. 1c). It has been previously shown that mitochondrial dysfunction in T2D reduces ATP production and Ca²⁺ signal generation causing defective GSIS. The involvement of VDAC was examined by overexpressing VDAC1 or suppressing VDAC2, which markedly inhibited GSIS in INS-1 cells (FIG. 1i, 1j; FIG. 2a, 2b). The manipulation of VDAC expression caused reciprocal variation in the other VDAC isoform (FIG. 1k, 1l). The decrease in GSIS may be attributed to mitochondrial dysfunction, since oxygen consumption rate (OCR) was inhibited both at basal (2.8 mM) and stimulated (16.7 mM) glucose (FIG. 2c, 2g). Prolonged exposure to high glucose concentrations impairs insulin secretion both in humans and in isolated islets (Boden G, et al. 1996. Am J Physiol 270: E251-258; Masini M, et al. 2014. Diabetes Res Clin Pract 104: 163-170). Of note, 72 h culture of INS-1 cells at 20 mM glucose reproduced the attenuation of OCR seen after VDAC1 overexpression or VDAC2 suppression (FIG. 2h, 2i). Impaired mitochondrial metabolism also explains the blunted increases in cytosolic and mitochondrial Ca², concentrations, crucial for GSI, in INS-1 cells with altered VDAC expression as well as under glucotoxicity (FIG. 2 j-k).

TABLE 2
Characteristics of organ donors

| Donor | | | | BMI | |
|---|---|---|---|---|---|
| Condition | Number | Gender | Age (year) | (index) | Hb1Ac (%) |
| ND | 82 | Male | 55.7 ± 1.5 | 23.7 ± 0.2 | 5.5 ± 0.04 |
| | 44 | Female | 58 ± 1.2 | 23.2 ± 0.34 | 5.6 ± 0.06 |
| IGT | 29 | Male | 62.1 ± 6.7 | 27.1 ± 4.1 | 6.2 ± 0.2 |
| | 17 | Female | 59.7 ± 6.2 | 27.2 ± 4.7 | 6.1 ± 0.1 |
| T2D | 22 | Male | 62.6 ± 1.9 | 26.5 ± 0.9 | 7.0 ± 0.3 |
| | 13 | Female | 59.8 ± 3.1 | 30.2 ± 1.0 | 6.8 ± 0.2 |

ND—non-diabetic;
IGT—impaired glucose tolerance;
T2D—Type 2 diabetes

The metabolic activity in human islet cells was also monitored by following the formation of formazan from tetrazolium salt (MTS), reflecting mitochondrial reductive capacity. In contrast to overexpression, knock-down of VDAC1 resulted in almost complete protection from glucotoxicity-induced lowering of reductive capacity. On the contrary, knock-down of VDAC2 impaired metabolism at 5 and 20 mM glucose (FIG. 2f), substantiating the essential role of VDAC2 in cell function. Next, cell death was investigated by measuring cytoplasmic nucleosomes in INS-1 cells. While forced overexpression of VDAC1 or VDAC2 down-regulation at 5 mM glucose did not significantly increase apoptosis, altered VDAC expression combined with the glucotoxic condition, 20 mM glucose, caused marked cell death (FIG. 2m). Thioredoxin interacting protein (TXNIP) is induced by oxidative stress and glucotoxicity through nuclear transfer and induction of carbohydrate response element-binding protein (ChREBP) (Shalev A. 2014. Mol Endocrinol 28: 1211-1220; Poungvarin N, et al. 2012. Diabetologia 55: 1783-1796). This effect is mimicked by the non-metabolisable glucose analogue 2-deoxyglucose (Shalev, 2014, ibid). T2D islets displayed increased transcripts of both ChREBP and TXNIP (FIG. 1e), confirming published results. As 2-deoxyglucose increased VDAC1 expression in INS-1 cells (data not shown), the involvement of TXNIP is likely. Moreover, knock-down of either ChREBP or TXNIP abrogated glucotoxicity-induced VDAC1 upregulation (FIG. 1f). These results demonstrate that VDAC1 induction is a consequence of glucose-mediated TXNIP activation. This prompted us to examine cellular VDAC1 localization.

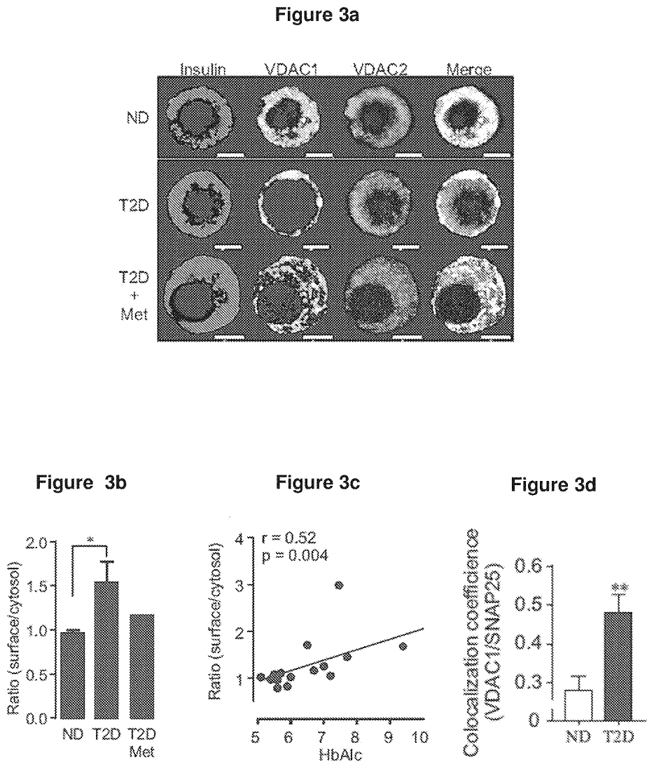

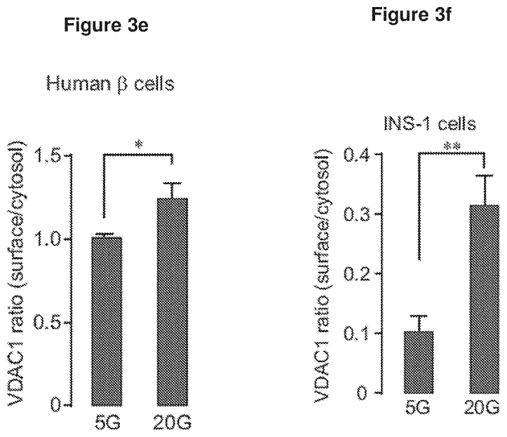

Extra-mitochondrial plasma membrane VDAC1 participates in volume regulation, ATP and metabolite transport as well as intrinsic mitochondrial apoptosis. Remarkably, the present invention demonstrates by confocal microscopy that VDAC1, but not VDAC2, surface expression occurs in T2D β-cells. In non-diabetic and in the single examined donor of four with documented metformin therapy, VDAC1 remained intracellular (FIG. 3a, 3b). VDAC1 surface expression correlated positively with HbA1c (FIG. 3c). High glucose culture of islets or INS-1 cells caused VDAC1 surface localization (FIG. 3a; FIG. 3e, 3f).

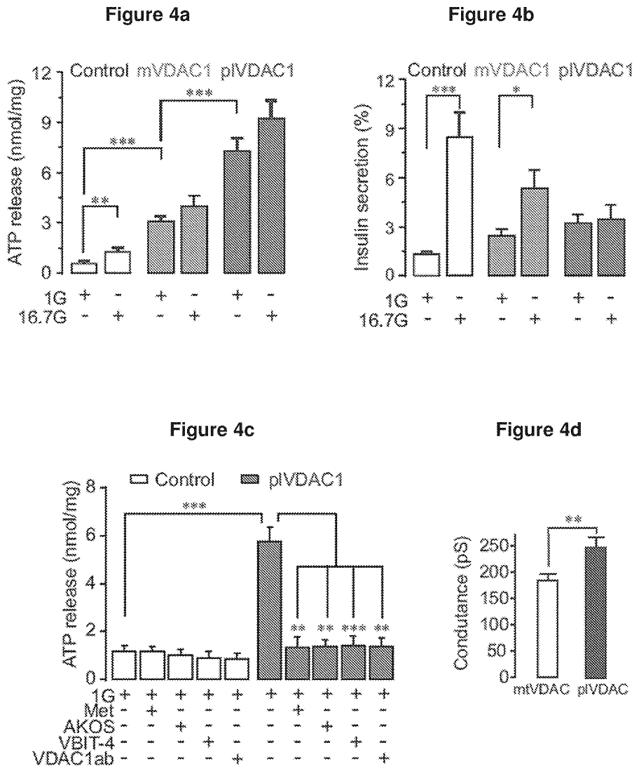

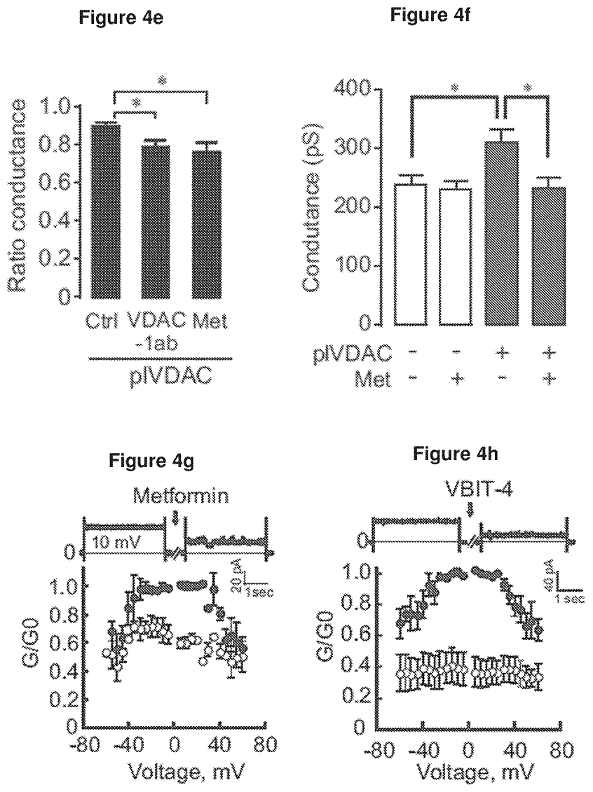

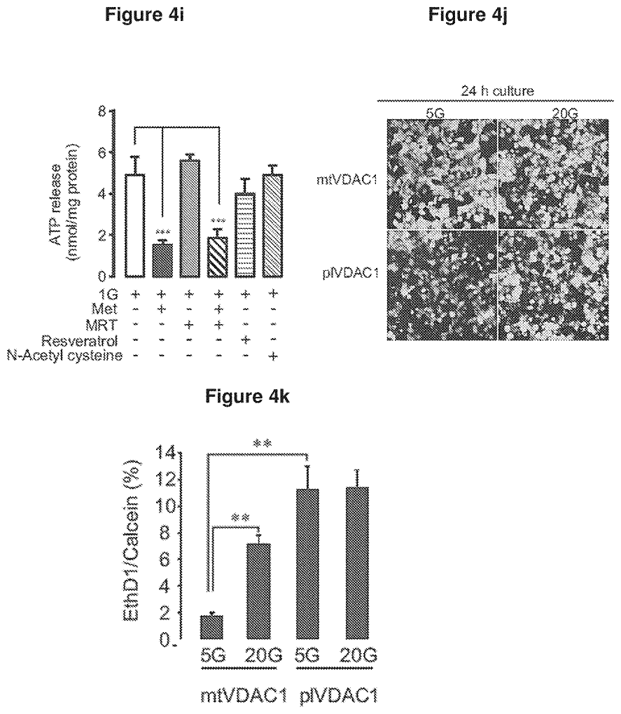

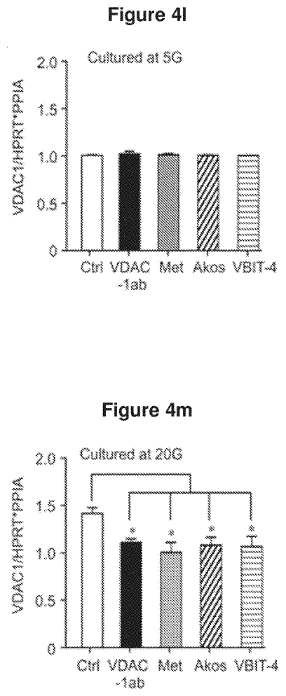

The functional consequence of aberrant VDAC1 subcellular localization was further studied. The mouse VDAC1 gene is alternatively transcribed, yielding exon1 splice variant encoding a plasma membrane-targeted protein (plVDAC1) (Buettner R, et al. 2000. Proc Natl Acad Sci USA 97: 3201-3206). Such splicing has not been reported for the human VDAC1 gene. In order to relate the present findings regarding exon-specific expression in rat and mouse to the human VDAC1 transcript and gene, the approximate sequences targeted by each primer were aligned to the human genome using BLAST. The targeted sequences were calculated using available data from manufacturers. Alignments to the human VDAC1 gene on chromosome 5 were then visualized using the Integrative Genomics Viewer. The exon positions of the human RefSeq mRNA (RefSeq ID NM_003374) were used. The rat plVDAC1 targeted region differs from rat mtVDAC1 by the inclusion of 13 b of intronic sequence before the start of exon 2 of human VDAC1, making splicing unlikely (data not shown). Nonetheless, plasma membrane resident VDAC1 has been documented in various mouse and human tissues, oriented with the mitochondrial surface residues facing the extracellular space. Opening of plVDAC1 initiated neuronal apoptosis, prevented by antibodies directed against the extracellular N-terminus of VDAC1. Metformin inhibits TXNIP activation by high glucose concentrations in both insulin-secreting cells (Shaked M, et al. 2011. PLoS One 6: e28804) and endothelial cells (Li X, et al. 2015. Mol Endocrinol 29: 1184-1194). Herein, 20 μM metformin were used, a concentration reported to inhibit hepatic glucose production (Madiraju A K, et al. 2014. Nature 510:542-546) and measured in patient plasma (Foretz M, et al. 2014. Cell Metab 20:953-966). In INS-1 cells, the VDAC inhibitory molecules VBIT-4 and AKOS (as well as N-terminal VDAC1 antibody and metformin) abrogated the glucose evoked VDAC1 upregulation. (FIG. 4l,4m).

plVDAC1 function in transfected INS-1 cells was further investigated. ATP, essential in GSIS (Wiederkehr A and Wollheim C B. 2012. Mol Cell Endocrinol 353: 128-137), was first measured. Overexpression of mitochondria targeted VDAC1 (mtVDAC1) resulted in a 3-fold increase in ATP release from the cells relative to control cells, compared to a 10-fold loss of ATP when cells were transfected with plVDAC1 (FIG. 4a). The robust GSIS in cells transfected with control plasmid was markedly reduced in mtVDAC1-transfected and completely abolished in plVDAC1-expressing cells (FIG. 4b). Moreover, glucose (20 mM) aggravated the marginal cell death in mtVDAC1 cells, while plVDAC1 was more harmful (FIG. 4j, 4k). The loss of cellular ATP was prevented by AKOS and VBIT-4 (having structural formula 1 and 10, respectively) as well as by VDAC1 N-terminal antibody and metformin (FIG. 4c). In patch-clamp experiments, INS-1 cells expressing plVDAC1 had 30% higher membrane conductance than cells transfected with mtVDAC1 (FIG. 4d). The increased conductance caused by plVDAC1 relative to control INS-1 cells was abolished by the acute addition of either VDAC1 antibody or metformin (FIG. 4e). Superfusion with metformin did not affect membrane currents in control INS-1 cells while abrogating the elevated conductance in plVDAC1-transformed cells (FIG. 4f). As mitochondrial and plasma membrane VDAC1 display identical amino acid sequences, patch clamp recordings were performed with purified mitochondrial VDAC1 protein reconstituted into planar lipid bilayers (Ben Hail et al., 2016, ibid). Metformin (30 μM) inhibited channel conductance by 50%.

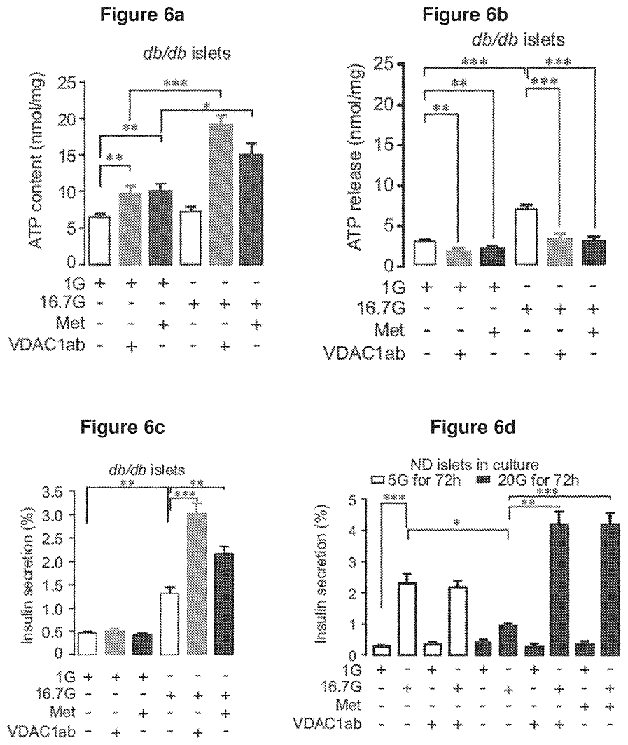

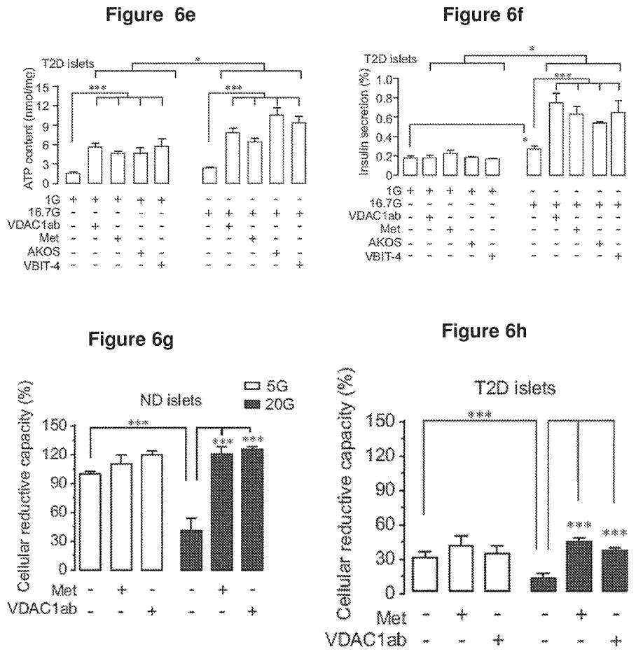

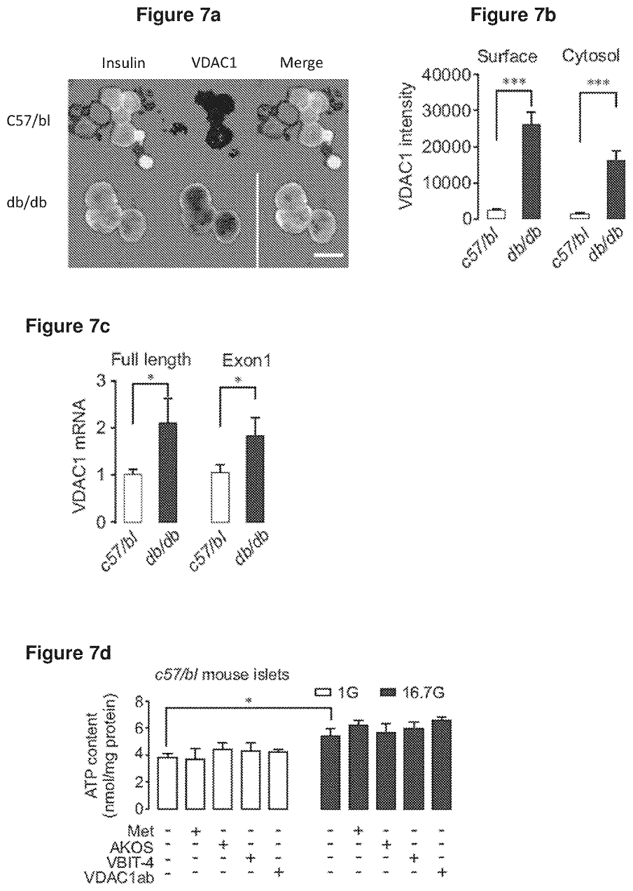

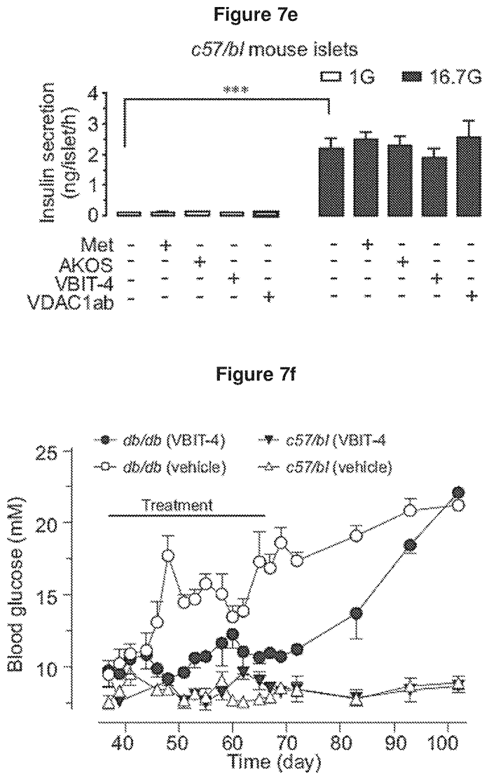

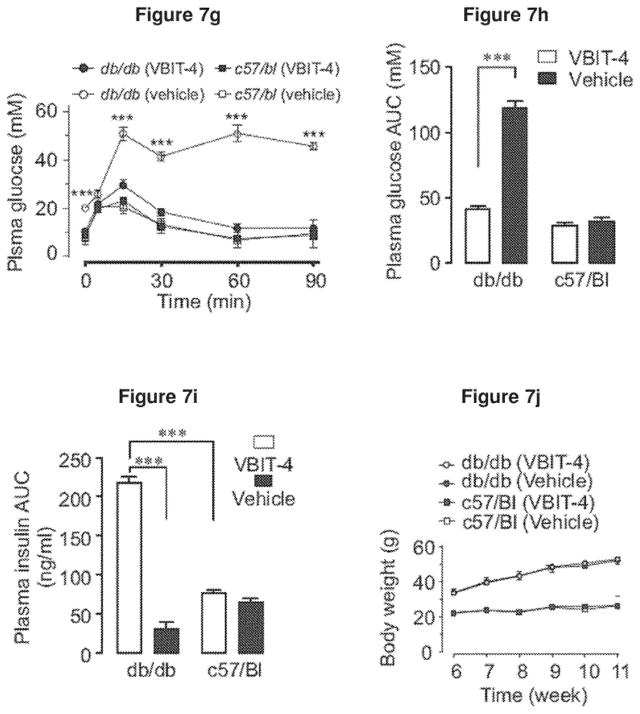

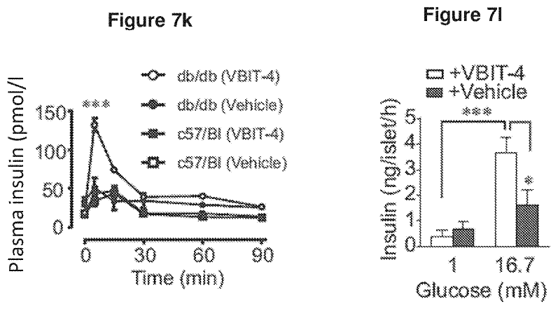

In db/db mice treatment with metformin attenuates the severity of hyperglycemia at onset of the disease (Cao K, et al. 2014. Free Radic Biol Med 67: 396-407). Islet cells from diabetic db/db mice were thus investigated. Like islets from T2D donors, β-cells from db/db but not C₅₇/bl mice showed surface expression of VDAC1 (FIG. 7a, 7b). This was associated with increased VDAC1 exon1 mRNA (FIG. 7c). Freshly isolated islets from hyperglycemic db/db mice displayed low ATP content, unaltered by 16.7 mM glucose (FIG. 6a). As expected from the surface localization, VDAC1 antibody or metformin raised ATP intracellular content and reduced the elevated ATP release (FIG. 6a, 6b). The inhibition of VDAC1 restored the stimulatory effect of glucose on ATP content (FIG. 6a) and markedly enhanced insulin secretion, which is attenuated in the db/db islets (FIG. 6c). Neither ATP content nor GSIS was affected by VDAC1 inhibition in islets from control mice (FIG. 7d, 7e), confirming VDAC1 dysfunction only in diabetic β-cells.

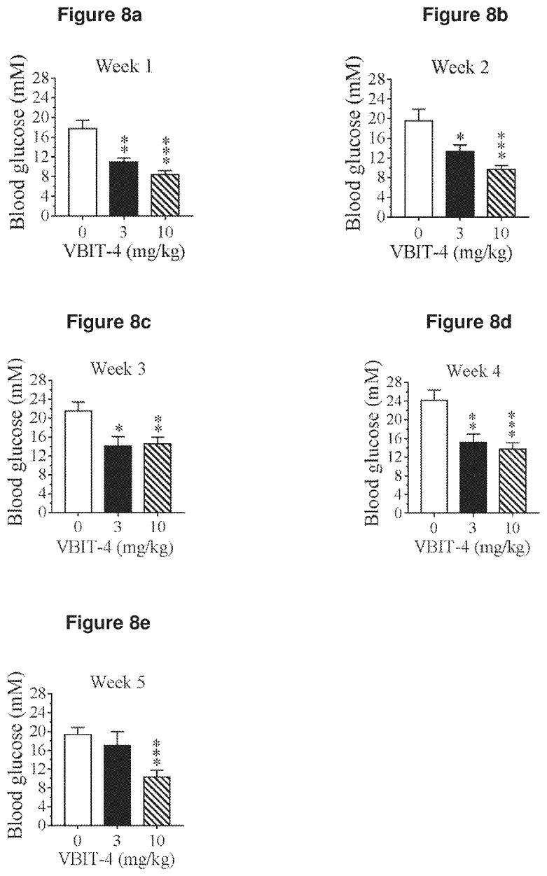

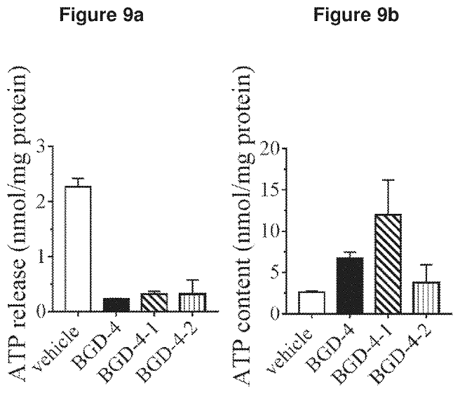

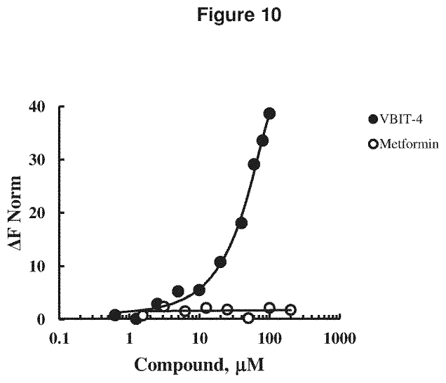

It was further investigated whether targeting cell membrane VDAC1 could also ameliorate β-cell function in human islets. Inhibition of VDAC1 using metformin, the VDAC1 piperazine- and/or piperidine-derivative inhibitors or antibodies had no effect on ATP content and cellular reductive capacity after culture at 5 mM glucose but markedly improved metabolism during glucotoxic conditions or in T2D islets (FIG. 7d; FIG. 6g, 6h). Inclusion of the antibody or metformin in the culture medium prevented the decline in GSIS observed in the islets cultured at 20 mM glucose, while secretion was unaltered under control conditions (FIG. 6d). Finally, daily injections of db/db mice with the VDAC1 inhibitor VBIT-4 prevented the development of severe hyperglycemia, which gradually reverted upon drug cessation (FIG. 7f). The drug markedly improved glucose tolerance and GSIS both in vivo and in isolated islets (FIG. 7h, 7i, 7k, 7l). The early administration of VBIT-4 also prevented increases in water consumption and urine production in the db/db mice but only had marginal effects in 14-week old animals with manifest hyperglycemia (data not shown). The compounds of the invention can be administered orally. As described in FIG. 8a-e, daily administration of VBIT-4 by gavage to db/db mice reduced the blood glucose concentration. At a dose of 10 mg/Kg body weight, the reduction was significant along the entire trail period (5 weeks).

In summary, the present invention shows that overexpression of mitochondrial VDAC1 and it's the protein aberrant translocation to the plasma membrane may explains the defective glucose-stimulated insulin secretion in T2D β-cells, due to loss of the essential metabolic coupling factor ATP through the membrane-localized VDAC1. The present invention further shows that the impaired reductive capacity and loss of ATP in the diabetic β cell is reverted by inhibition of VDAC1, particularly by the derivatives VIBT-4 and AKOS (having formula 1 and 10, respectively) and VDCA1 specific antibody leading to restoration of insulin secretion and prevention of hyperglycemia in diabetic mice. Remarkably, inhibition of VDAC1 had no effects in control mice or in islets from non-diabetic human donors (data not shown), strongly suggesting that only the membrane localized VDAC1 is sensitive to the intervention. Therapeutic concentrations of metformin also ameliorate insulin release through the prevention of VDAC1 induction and direct inhibition of VDAC1 conductance, although binding of metformin to VDCA1 was not observed in the microscale thermophoresis assay described in the Example section hereinafter.

### Pharmaceutical Compositions

Although the VDAC1 inhibitory piperazine and/or piperidine derivatives of the present invention can be administered alone, it is contemplated that these compounds will be administered in a pharmaceutical composition containing the VDAC inhibitory compounds of the invention together with a pharmaceutically acceptable carrier or excipient.

The pharmaceutical compositions of the present invention can be formulated for administration by a variety of routes including oral, transdermal, parenteral (subcutaneous, intraperitoneal, intravenous, intraarterial, transdermal and intramuscular), topical, intranasal, or via a suppository. According to certain exemplary embodiments, the compositions are formulated for oral administration. According to additional certain exemplary embodiments, the compositions are formulated for parenteral administration. Such compositions are prepared in a manner well known in the pharmaceutical art and comprise as an active ingredient at least one compound of the present invention as described hereinabove, and a pharmaceutically acceptable excipient or a carrier. The term “pharmaceutically acceptable” means approved by a regulatory agency of the Federal or a state government or listed in the U.S. Pharmacopeia or other generally recognized pharmacopeia for use in animals and, more particularly, in humans.

During the preparation of the pharmaceutical compositions according to the present invention the active ingredient is usually mixed with a carrier or excipient, which may be a solid, semi-solid, or liquid material.

For intravenous administration, the compounds of the invention can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

The compositions can also be formulated for oral administration, e.g., in the form of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

Any method can be used to prepare the pharmaceutical compositions. Solid dosage forms can be prepared by wet granulation, dry granulation, direct compression and the like.

The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In one embodiment, the active ingredient is dissolved in any acceptable lipid carrier (e.g., fatty acids, oils to form, for example, a micelle or a liposome).

Compositions for inhalation include solutions and suspensions in pharmaceutically acceptable aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, controlled-release formulations and the like, as are known in the art.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

A compound of the present invention can be delivered in an immediate release or in a controlled release system. In one embodiment, an infusion pump may be used to administer a compound of the invention, such as those used for currently known treatment of diabetes (e.g. insulin administration). In certain embodiments, a compound of the invention is administered in combination with a biodegradable, biocompatible polymeric implant, which releases the compound over a controlled period of time. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.).

Furthermore, the pharmaceutical compositions may be formulated for parenteral administration (subcutaneous, intravenous, intraarterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

The amount of a compound of the invention that will be effective in the treatment of a particular condition of diabetes will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the diabetic condition, and should be decided according to the judgment of the practitioner and each patient's circumstances. Effective doses may be extrapolated from dose-response curves derived from in vitro or animal model test bioassays or systems. According to certain embodiments, the doses can be extrapolated from the dose effective in treating mice. According to certain embodiments the mice dose is from 0.5-100, 1-70, 5-50 or 10-40 mg/Kg mouse body weight. Each possibility represents a separate embodiment of the present invention

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Materials and Methods

### Reagents and Kits

Fatty acid free bovine serum albumin (Boehringer, Germany), Rabbit polyclonal anti-VDAC1 antibody (N-terminal) (Abcam), monoclonal anti-VDAC1 antibody (N-terminal) (Calbiochem and Abcam), goat polyclonal anti-VDAC2 (Abcam), HRP-conjugated antigoat and anti-rabbit IgG (Santa Cruz Biotechnologies), insulin radioimmunoassay kit (Millipore), insulin ELISA kit (Mercordia, Sweden), siRNAs of VDAC1, VDAC2, ChREBP and TXNIP (Ambion), human VDAC1 and VDAC2 lentivirus shRNA as well as the primers for ChREBP and TXNIP detection (Santa Cruz Biotechnology), Vdac1 plasmid construct (Source BioScience imagenes), The multiple kinase inhibitor (MRT1) is described in reference (Clark, K. et al. 2012. Proceedings of the National Academy of Sciences of the United States of America 109, 16986-16991), resveratrol (AK Scientific Inc). Plasma membrane lead VDAC1 (pVDAC1) and mitochondrial VDAC1 (mtVDAC1) plasmid constructs were kind gifts from Dr. R. Beuttner. AKOS-022075291 (also designated AKOS-022 or AKOS was purchased from AKos Consulting & Solutions (Germany). VBIT-4 molecules were synthesized by ChemPartner (Chengdu, China).) All other chemicals were from Merck AG, (Darmstadt, Germany) or Sigma (USA).

### Cell Culture

INS-1 832/13 cells (kindly donated by Dr. C. B. Newgaard, Duke University, USA) were cultured in RPMI-1640 containing 11.1 mM D-glucose and supplemented with 10% fetal bovine serum, 100 U/ml penicillin (Gibco), 100 g/ml streptomycin (Gibco), 10 mM HEPES, 2 mM glutamine, 1 mM sodium pyruvate, and 50 M β-mercaptoethanol (Sigma), at 37° C. in a humidified atmosphere containing 95% air and 5% CO₂. For VDAC1 over-expression by VDAC1 plasmid (1 μg/ml) and VDAC2 down-regulation by and siRNA, INS1 832/13 cells were cultured to 60% confluence. To study the long-term effects (24-72 h) of high glucose (20 mM) (glucotoxicity) compared to basal glucose (5 mM), INS-1 832/13 cells were cultured with RPMI 1640 complete media (Ahmed, M., et al., 2010. Islets 2, 283-292) in the presence or absence of indicated agents and thereafter VDAC1 expression, cell viability and function was measured.

### Plasmid and Transient Transfections

Full-length cDNA encoding prevalidated Vdac1 construct was purchased from Source BioScience imagenes (pDEST26), Berlin. INS1 832/13 cells were seeded in six-well plates at a density of ˜5×10⁵ cells in culture media without antibiotics and transfected with Effectene Transfection Reagent (Qiagen) according to the manufacturer's instructions. Cells were transfected for 24 h with the plasmid VDAC1 at a final concentration of 1 μg/ml or control plasmid (non-targeting) at the same concentration before changing to fresh media including antibiotics. At 48 h after transfection, the cells were harvested and analyzed by immunoblotting and qPCR for the relative level of various proteins and genes. Transient transfection assays using the luciferase reporter gene were carried out using the standard calcium phosphate precipitation method (Mahon, M. J. 2011. BioTechniques 51, 119-128).

### Insulin Secretion in Cultured INS-1 832/13 Cells

Cells with VDAC1 over-expressed or VDAC2 down-regulated were used to measure the insulin secretion. To this end, INS-1 cells were kept in HEPES balanced salt solution (HBSS; secretion assay buffer, SAB, 114 mM NaCl; 4.7 mM KCl; 1.2 mM KH₂PO₄; 1.16 mM MgSO₄; 20 mM HEPES; 2.5 mM CaCl₂; 25.5 mM NaHCO₃; 0.2% BSA, pH 7.2) supplemented with 2.8 mM glucose for 2 h at 37° C. Thereafter the cells were incubated for 1 h in the same medium with the denoted glucose concentrations and test agents. After incubation an aliquot of media was removed for analysis of insulin.

### Conductance Measurement by Patch Clamp

Whole-cell currents in INS-1 832/13 cells were evoked and recorded by EPC10 amplifier and Pulse software (HEKA, Lambrecht/Pfalz, Germany) as previously described (Buda P. et al. 2013. PLoS One. 8(5):e64462) with the temperature maintained at 32° C. The cells were continuously perfused with extracellular solution containing 118 mM NaCl, 20 mM tetraethylammonium chloride, 5.6 mM KCl, 2.6 mM CaCl2), 1.2 mM MgCl2, 5 mM HEPES and 5 mM glucose (pH 7.4 with NaOH) in the presence of 100 μM tolbutamide (T0891, Sigma-Aldrich). The intracellular solution consisted of 125 mM Cs-glutamate, 10 mM CsCl, 10 mM NaCl, 1 mM MgCl2, 5 mM HEPES, 3 mM Mg-ATP, 0.1 mM cAMP and 0.05 mM EGTA (pH 7.2 with CsOH). 20 μM metformin was used for perfusion, and approximately 8 mM metformin or 130 nM VDAC1 antibody (both diluted approximately 100 fold) for acute addition as indicated in figure legends or text. Conductance was measured by applying 200-ms voltage ramps from −90 mV to −50 mV. In the experiments studying the effects of acute addition of VDAC1 ab or metformin, conductance was measured continuously in the same single cell before, during and after the acute addition of the respective compounds (VDAC1 antibodies or metformin). Data presented are from 15 s after the addition when steady-state was reached.

For experiments in FIG. 4F, the cell dish in the experimental chamber was continuously exposed to 20 μM metformin. Conductance measurements were performed after at least 5 minutes exposure, and the cell dish was replaced no later than after 1 hour of perfusion. No wash-out experiments were performed in this experiment.

### VDAC Current Measurement on the Reconstituted VDAC1 in Planar Lipid Bilaver (PLB)

VDAC1 purified from rat liver mitochondria as described previously (Ben-Hail D et al. 2016. J Biol Chem 2016; 291, 24986-25003). To measure single and multiple channel current, a planar lipid bilayer PFLB was prepared from soybean asolectin dissolved in n-decane (30 mg/ml). Purified VDAC1 (1-100 ng) was added to the chamber defined as the cis side containing 0.5 M NaCl. After one or a few channels were inserted into the PLB, excess protein was removed by perfusing the cis chamber with ˜10 volumes of solution to prevent further channel incorporation. Following several recordings of channel activity at different voltages, metformin or VBIT-4 was added to the cis chamber, and currents through the channel were again recorded. Currents were recorded by voltage-clamping using a Bilayer Clamp BC-535B amplifier (Warner Instruments, Hamden, Conn.). Currents were measured with respect to the trans side of the membrane (ground). The currents were low pass-filtered at 1 kHz and digitized online using a Digidata 1440-interface board and Clampex software (Axon Instruments, Union City, Calif.). Analysis was done using pClamp 10.2 software (Axon Instruments, Union City, Calif.), or excel (Microsoft).

### Animals

5 weeks old db/db mice and control (C57/bl) (Janvier Laboratory, France), weighing 18-25 g, were used throughout the experiments. They were given a standard pellet diet (B&K) and tap water ad libitum. The experimental procedures were approved by the Ethics Committee for Animal Research at Lund University. Isolation of pancreatic islets was performed by retrograde injection of a collagenase solution via the bile-pancreatic duct and islets were then collected under a stereomicroscope at room temperature.

### Intraperitoneal Glucose Tolerance Tests (IPGTT).

IPGTTs were performed in db/db and c57/bl mice after treatment with VDAC1 blocker (daily intraperitoneal injection with VBIT-4, 25 mg/5025 body weight) for 5 weeks. Prior to IPGTT test, the mice were fasted for 4 h. Glucose was dissolved in 0.9% NaCl and 2.0 g glucose/kg body weight was injected intraperitoneally. Serial blood sampling thereafter from vena saphena was performed at 0, 5, 15, 30 and 90 min as previously described elsewhere 35. Blood glucose was analyzed using glucose oxidase method and plasma insulin was analyzed by ELISA (Salehi, A. et al. 2008. PLoS ONE 3, e2165; Rosengren, A. H. et al. 2010. Science 327, 217-220). Total volume load was 0.3 ml. The Cumulative (area under the curve) changes in plasma glucose or insulin were calculated by subtracting the recorded values from basal (time 0).

### Human Pancreatic Islets

Human pancreatic islets were obtained through collaboration between Human Tissue Laboratory within Lund University Diabetes Centre (LUDC) and the Nordic Network for Clinical Islet Transplantation (Prof. Olle Korsgren, Uppsala University, Sweden). Donors were grouped according to HbA1c i.e. less than 6% (ND), between 6% and 6.5% (IGT), higher than 6.5% or history of diabetes (T2D). The human islets (70-90% purity) had been cultured in CMRL 1066 (ICN Biomedicals, Costa Mesa, Calif.) supplemented with 10 mM HEPES, 2 mM L-glutamine, 50 g/ml gentamicin, 0.25 g/ml fungizone (Gibco, BRL, Gaithersburg, Md.), 20 μg/ml ciprofloxacin (Bayer Healthcare, Leverkusen, Germany) and 10 mM nicotinamide at 37° C. (5% CO₂) for 1 to 5 days prior to the arrival in the laboratory. The islets were then hand-picked under stereomicroscope prior to use. All procedures using human islets were approved by the ethical committees at Uppsala and Lund Universities, Sweden.

### Glucose-Stimulated Insulin Secretion (GSIS) in Human and Mouse Islets

Human pancreatic islets were collected under a stereomicroscope at room temperature and cultured at 5.5 or 20 mM glucose in the absence or presence of test agents for 72 h. Thereafter the islets were washed and preincubated for 30 min at 37° C. in Krebs Ringer bicarbonate buffer, pH 7.4, supplemented with N-2 hydroxyethylpiperazine-N′-2-ethanesulfonic acid (10 mM), 0.1% bovine serum albumin, and 1 mM glucose. Each incubation vial contained 12 islets in 1.0 ml KRB buffer solution and treated with 95% O₂ and 5% CO₂ to obtain constant pH and oxygenation. After preincubation, the buffer was changed to a medium containing either 1 mM or 16.7 mM glucose. The islets were then incubated for 1 h at 37° C. in a metabolic shaker (30 cycles per min). Immediately after incubation an aliquot of the medium was removed for analysis of insulin and the islets were incubated in acid-ethanol for insulin content determination by radioimmunoassay.

Quantitative Polymerase Chain Reaction (qPCR)

Total RNA from handpicked mouse islets, human donor islets (Diabetic and non-diabetic) or INS1 832/13 cells were extracted using RNAeasy (Qiagen, Hilden, Germany). RNA (0.5 g) was used for cDNA synthesis with SuperScript (Invitrogen, Carlsbad, Calif., USA). Concentration and purity of total RNA was measured with a NanoDrop ND-1000 spectrophotometer (A260/A280>1.9 and A260/A230>1.4) (NanoDrop Technologies, Wilmington, Del.) and RNA Quality Indicator (RQI) higher than 8.0 (Experion Automated Electrophoresis, Bio-Rad, USA) was considered to be high-quality total RNA preparations. A 10 μl of reaction mixture with 20 ng cDNA, 5 μl TaqMan mastermix (Applied Biosystems, Foster City, Calif., USA), and 100 nM TagMan gene expression assay were run in a 7900HT Fast Real-Time System (Applied Biosystems). The qPCR was carried out as follows: 50° C. for 2 minutes, 95° C. for 10 minutes, 40 cycles of 95° C. for 15 seconds, and 60° C. for 1 minute. The amount of mRNA was calculated relative to the amount of housekeeping genes (GAPDH, PPIA or HPRT) mRNA in the same sample by the formula X0/R0=2CtR-CtX, where X0 is the original amount of mRNA for the gene of interest, R0 is the original amount of HPRT mRNA, CtR the Ct value for HPRT, and CtX the Ct value for the gene of interest. Primer sequences are provided upon request.

To measure the expression level of VDAC1 and VDAC2 under glucotoxic (20 mM glucose) compared to normal condition (5 mM glucose), human islets (300 islets/30 mm Dish) were cultured in RPMI 1640 medium containing either 5 or 20 mM glucose with or without indicated test agents in a humidified incubator (37° C., 5% CO₂) for 24 to 72 h. VDAC1 and VDAC2 mRNA and protein level were investigated by both qPCR and Western blotting relative to the expression of GAPDH and β-actin respectively.

### Immunoblotting

Human islet or INS1 832/13 cells were suspended in 100 μl of SDS-buffer (50 mM Tris-HCl, 1 mM EDTA) supplemented with Complete protease inhibitor cocktail (Roche), frozen and sonicated on ice on the day of analysis. The protein content of the homogenates was determined according to the Bradford method (Bradford, M. M. A 1976. Analytical biochemistry 72, 248-254). Homogenate samples of islets and INS1 832/13 representing 30 μg of total protein were run on 7.5% SDS-polyacrylamide gels (Bio-Rad, Hercules, Calif., USA). After electrophoresis, proteins were transferred to nitrocellulose membranes (Bio-Rad, Hercules, Calif., USA). The membranes were blocked in LS-buffer (10 mM Tris, pH 7.4, 100 mM NaCl, 0.1% Tween-20) containing 5% non-fat dry milk powder for 40 min at 37° C. Subsequently the membranes were incubated over night with rabbit-raised polyclonal anti-VDAC1 and goat-raised polyclonal anti-VDAC2 antibodies (1:500) at room temperature. After washing (three times) in LS-buffer the membranes were finally incubated with horseradish peroxidase-conjugated anti-goat and anti-rabbit antibodies (1:10,000). Immunoreactivity was detected using an enhanced chemiluminescence reaction (Pierce, Rockford, Ill., USA).

### Immunostaining and Confocal Imaging.

Isolated human or mouse islets as well as INS-1 cells were seeded on the glass-bottom dish cultured overnight. Cells were then washed twice and fixed with 3% PFA for 10 min, followed by permeabilization with 0.1% Triton-X 100 for 15 min. The blocking solution contained 5% normal donkey serum in PBS and was used for 15 min. Primary antibodies against mouse VDAC1 (Abcam), goat VDAC2 (Abcam) and Guinea pig insulin (Eurodiagnostica) were diluted in blocking buffer and incubated overnight at 4° C. Immunoreactivity was quantified using fluorescently labeled secondary antibodies (1:200) and visualized by confocal microscopy (Carl Zeiss, Germany). The ratio is calculated by mean intensity of plasma membrane to mean intensity in cytosol, according to the formula:

$$\frac{[ \frac{( i 1 * a 1 ) - ( i 2 * a 2 )}{a 1 - a 2} ]}{[ \frac{( i 2 * a 2 ) - ( i 3 * a 3 )}{a 2 - a 3} ]}$$

where i1, i2 and i3 represent the intensities of whole cell, cytosol and nucleus, a1, a2 and a3 represent the area of whole cell, cytosol and nucleus respectively (Buda et al. 2013, ibid).

### Single Cell ATP/ADP Ratio Measurement

For single cell ATP/ADP ratio measurements, INS-1 cells were co-transfected as above with either wtVDAC1 or des-Cys(127/232)VDAC1 plasmid together with PercevalHR (1 μg/ml) each. Single cell imaging was performed by confocal microscopy as described (Berg J, et al., Nature methods. 2009; 6(2):161-6) and applied to INS-1 cells (Ofori J K, et al., Scientific reports. 2017; 7:44986). Expression levels were determined by qPCR.

### TIRF Microscopy

INS-1 cells were seeded on glass-bottom dishes and transfected with wild-type VDAC1-EGFP and des-Cys(127/232)VDAC1-EGFP for 48 hours. The membrane expression of wild type and desCys(127/232)VDAC1 was measured by TIRF imaging, which detects the VDAC1 signal about 150 nm close to the glass surface. The analysis of VDAC1 spots was performed by ImageJ Plugin and ZEN2012 software. The experiments were repeated 3 times with 30 cells in each group of wild type and des-Cys(127/232)VDAC1 transfected cells.

Small Interfering RNA (siRNA) for Silencing

For VDAC1 and VDAC2 small interfering RNA (siRNA) experiments, 20-25 nucleotide stealth prevalidated siRNA duplex designed for rat Vdac1 and Vdac2 (Applied Biosystem) were used. INS1 832/13 cells were seeded in six-well plates at a density of ˜5×10⁵ cells in culture media without antibiotics and transfected with DharmaFECT® 1 (Dharmacon; Lafayette, Colo., USA) according to the manufacturer's instructions. Cells were transfected for 24 h with the Vdac1 and Vdac2 siRNA at a final concentration of 50 nM or with control siRNA (non-targeting siRNA) at the same concentration before changing to fresh media including antibiotics. At 72 h after transfection, cells were lysed to extract total RNA or protein to measure the knockdown efficacy.

For silencing of Chrebp and Txnip in. INS1 832/13 cells, 20-25 nucleotide stealth prevalidated siRNA duplex designed for rat Chrebp and Txnip (Applied Biosystem) were used and the same protocol as for VDAC silencing was followed. At 72 h after transfection, cells were lysed to extract total RNA or protein to measure Chrebp and Txnip knockdown efficiency as well as Vdac1 expression.

Silencing by shRNA Mediated (Lentivirus) in Human Islets

Specific silencing of endogenous human hVDAC1 or hVDAC2 was achieved using lentiviral based ShRNA-silencing technique (Santa Cruz, Calif., USA). Isolated human islets were incubated at 2.8 mM glucose plus Polybree for 90 min. Thereafter the medium was removed and the islets were washed before addition of culture medium with lentiviral particle containing VDAC1-shRNA or VDAC2-shRNA (5 μl/ml) and the islets were cultured for 72 h at 5 or 20 mM glucose. For comparison a scramble (lentiviral particles without targeting any specific region) served as control. After the culture period the medium was removed and the islets were dispersed into single cells and subjected to cell viability assay by MTS.

### Measurement of Cellular Reductive Capacity (MTS), Apoptosis and Viability

The reductive capacity of cells was measured either on INS-1 cells or dispersed human islet cells when the INS-1 cells or islets were subjected to 5 or 20 mM glucose for 72 h in the absence or presence of test agents or after down-regulation of VDAC1 and VDAC2 as described elsewhere (35, 40). Measurement of reductive capacity was performed using the MTS reagent kit according to the manufacturer's instructions (Promega). Apoptosis was measured with the Cell Death Kit (Roche Diagnostics), which quantifies the appearance of cytosolic nucleosomes.

To measure the cell viability in living cells, EthD1 (Ethidium homodimer-1) and calcein were used to indicate death and live cell in INS-1 cells, respectively according to manufacturer (ThermoFisher, USA). The plasma membrane targeted VDAC1 (plVDAC1) and mitochondrial VDAC1 (mtVDAC1) were overexpressed in INS-1 cells cultured with either 5 mM glucose (5G) or 20 mM glucose (20G). The mean intensity of Ethidium and Calcein were calculated to indicate live and death cells, respectively.

### Detection of Oxygen Consumption Rate (OCR).

OCR was measured in INS-1 832/13 cells using the XF (extracellular flux) analyser XF24 (Seahorse Bioscience), as previously described in detail (Wiederkehr et al. 2011. 13, 601-611). An assay medium composed of (in mM) 114 NaCl, 4.7 KCl, 1.2 KH₂PO₄, 1.16 MgSO₄, 20 HEPES, 2.5 CaCl₂, 0.2% bovine serum albumin, pH 7.2, and supplemented with 2.8 mM glucose was used in the XF analysis. The cells were seeded in an XF24 24-well cell culture microplate at 2.5×10⁵ cells/well (0.32 cm² growth area) in 500 μl of growth medium and incubated overnight at 37° C. in a humidified atmosphere of 95% air and 5% CO₂. Prior to assay, RPMI 1640 medium was removed and replaced by 750 μl of assay medium. The cells were preincubated under these conditions for 2 h at 37° C. in air. The experiments were designed to determine respiration in low (2.8 mM) glucose and for 60 min following the transition to high (16.7 mM) glucose. The proportions of respiration driving ATP synthesis and proton leak were determined by the addition of oligomycin (4 g/ml). After a further 30 min, 4 M of dinitrophenol was added to determine maximal respiratory capacity. After a further 10 min, 1 M rotenone was added to block transfer of electrons from complex I to ubiquinone.

### ATP Determination

ATP release from isolated mouse or human islets or from INS-1 cells after transfection with mitochondrial targeted VDAC1 (mtVDAC1) or plasma membrane targeted VDAC1 (plVDAC1) plasmids was determined using a luminometric assay kit according to manufacturer's recommendation (Biovision). After incubation of islets (50/vial) or INS-1 cells for 60 min, an aliquot of the media was removed for subsequent measurements of released ATP. Then the islets or INS-1 cells were washed 3 times and the lysates were used for measurements of ATP and protein contents followed the protocol provided by the vendor.

### Mitochondrial and Cytosolic Ca²⁺ Imaging in Single Cells

INS-1 cells were seeded on 24-well plates and grouped into 4 conditions: Control with 5 mM glucose, overexpressed (OE) by VDAC1 and silencing (KD) of VDAC2 by siRNA in 5 mM glucose culture, 3 days treated with 20 mM glucose. 24 hours prior to Ca²⁺ imaging, the cells were transferred to glass-bottom dishes by 1:6 (1×10⁵ cells). The cells were stained 1 h with Rhod-2 (0.75 uM) and Fluo-5F (0.5 μM) dissolved in the perfusion buffer (KRB). Time lapse ROI images were acquired by confocal and the mean intensity of ROIs was analyzed by ZEN 2009 software. The data calculation was performed with Excel and normalized ratio was calculated by Fi/FO (Mahon 2011, ibid).

### Statistics

The results are expressed as means±SEM for the indicated number of observations or illustrated by an observation representative of a result obtained from different experiments (Western blots). The significance of random differences were analyzed by Student's t-test or where applicable the analysis of variance followed by Tukey-Kramers' multiple comparisons test. P value <0.05 was considered significant.

### Example 1: VDAC Expression and Function in β-Cells

FIGS. 1 and 2 depict the expression and function of VDAC in β-cells. In islets from T2D organ donors, VDAC1 mRNA was upregulated, while VDAC2 was repressed (FIG. 1a). Similar changes occurred at the protein level (FIGS. 1g and 1h). FIG. 1b shows that VDAC1 mRNA expression correlates with the preterminal blood glucose as reflected by HbA1c in ND (HbA1c<6.0%) donors. There was a borderline significance when all donor islets were tested including 15 T2D (n=30, R²=0.27; P<0.049) (not shown). The insert shows islet VDAC1 mRNA in ND (n=15), in T2D including metformin treated donors (n=15) and T2D with documented metformin therapy (n=4). (*p<0.05, **P<0.001 by Anova). Islets from T2D donors with documented metformin therapy did not display elevated VDAC1 mRNA. In contrast, culture of human islets under glucotoxic conditions, reproduced the T2D profile, increasing VDAC1 and decreasing VDAC2 mRNA (FIG. 1c). FIG. 1d shows the expression of VDAC1 in non-diabetic human islets cultured for 72 h at 5 and 20 mM glucose in the presence or absence of metformin (Met, 20 μM). Inclusion of 20 μM metformin in the 20 mM glucose culture prevented the increase in VDAC1 mRNA (FIG. 1d). These results link metformin action to VDAC1 gene expression. Next, the impact of the T2D VDAC expression profile was investigated by overexpressing VDAC1 and silencing VDAC2 in INS-1 clonal β-cells, reaching similar mRNA levels as in T2D islets (FIG. 1i, 1j). This caused reciprocal alteration in the other VDAC isoform, i.e. VDAC1 induction suppressed VDAC2 expression (FIG. 1k, 1l).

It is well documented that glucotoxicity, like oxidative stress, upregulates thioredoxin interacting protein (TXNIP) by nuclear transfer and induction of carbohydrate response element-binding protein (ChREBP). This genetic programming is also activated by the non-metabolizable glucose analogue 2-deoxyglucose. T2D islets displayed increased transcripts of both ChREBP and TXNIP (FIG. 1e), confirming published results. VDAC1 induction by 2-deoxyglucose in INS-1 cells substantiates the involvement of TXNIP in VDAC1 gene regulation by glucotoxicity. Moreover, knock-down of either ChREBP or TXNIP abrogate glucotoxicity-induced VDAC1 upregulation (FIG. 1F).

GSIS was markedly inhibited after VDAC1 overexpression or VDAC2 knock-down (FIG. 2a, 2b). The blunted GSIS was due to mitochondrial dysfunction, since oxygen consumption rate (OCR) was inhibited in cells with altered VDAC isoform expression (FIG. 2c, 2g). Cell culture under glucotoxic condition caused a similar attenuation of OCR (FIG. 2h, 2i). The impaired metabolism resulted in blunted glucose-induced rises in cytosolic and mitochondrial Ca²⁺ during acute stimulation (FIG. 2j, 2k, 2l). Both of these signals are crucial for GSIS. VDAC1 induction mediates glucotoxicity, as the abrogated ATP generation and impaired GSIS after 72 h at 20 mM glucose were prevented by VDAC1 knock-down in human islets. In contrast, VDAC2 suppression reduced basal islet ATP and prevented both ATP generation and stimulated insulin secretion (FIG. 2d, 2e). Defective mitochondrial metabolism was also evident, when islet cell overall reductive capacity was monitored (FIG. 2f). Thus, VDAC1 expression impacts negatively on cellular ATP levels and insulin secretion.

Evaluation of cell death by determining cytoplasmic nucleosomes in INS-1 cells after overexpression of VDAC1 or VDAC2 down-regulation at 5 mM glucose did not show significantly increased apoptosis. In contrast, altered VDAC1 expression combined with 20 mM glucose, caused marked cell death (FIG. 2m).

Metformin has been shown to prevent TXNIP induction by glucotoxicity in both insulin-secreting and insulin-sensitive endothelial cells. The results demonstrate that VDAC1 overexpression is a consequence of glucose-mediated TXNIP induction, a mechanism that is prevented by metformin. Moreover, defective ATP generation rather than apoptosis explains the impaired GSIS. This prompted us to examine whether altered localization of overexpressed VDAC1 underlies the defective beta cell function.

### Example 2: VDAC1 Expression and Localization in Human β-Cells

FIG. 3 illustrates the localization of VDAC1 in human β-Cells. FIG. 3a is immunofluorescence images of VDAC1 and VDAC2 in human islet β-cells cultured at 5 or 20 mM glucose (5G and 20G, respectively) for 72 h as well as in β-cells from T2D donors of which one had received metformin therapy. Note VDAC1 expressed prominently on the β-cell surface at 20G or in T2D islets. FIG. 3b shows β-cell surface expression of VDAC1 given as ratio of surface/cytosolic VDAC immunofluorescence intensity in islets of ND or T2D (n=8 donors each) as well as one T2D donor with documented metformin therapy. FIG. 3c shows that VDAC1-cell surface expression correlates with HbA1c values in islets of 15 T2D donors and non-diabetic islet donors. To verify the surface localization of VDAC1, double immunofluorescence staining was performed in pancreas sections of non-diabetic and T2D donors. VDAC1 was clearly overexpressed at the T2D beta cell surface, shown by the co-localization with the plasma membrane-associated SNARE protein SNAP-25 (FIG. 3d). FIGS. 3e and 3f show the ratio of surface/cytosolic VDAC1 immunofluorescence intensity in islets of human beta cells and INS-1 cells in different glucose conditions. VDAC1 localization to the cell surface is increased in high glucose. Thus, VDAC1 mistargeting to the plasma membrane may impair GSIS in T2D.

### Example 3: The Effect of VDAC1 Cell Surface Expression on ATP Handling, Insulin Secretion Membrane Conductance, and Cell Viability

To further substantiate the consequence of aberrant VDAC1 subcellular localization and overexpression, ATP levels during 1 h-experiments in plVDAC1-expressing INS-1 cells were monitored. Overexpression of wild type VDAC1 (mtVDAC1) resulted in a 3-fold increase in ATP release from the cells, suggesting mistargeting of VDAC1 to the plasma membrane. This was validated by plasma membrane targeted VDAC1 (plVDAC1) expression, which caused a 10-fold ATP loss (FIG. 4a). FIG. 4a shows ATP release after 1 h incubation at 1 (1G) or 16.7 mM glucose (16.7G) from INS-1 cells transfected with either mtVDAC1 or plVDAC1. For comparison, vector transfected controls are also shown. The robust GSIS in cells transfected with control plasmid was markedly reduced in mtVDAC1-transfected and completely abolished in plVDAC1-expressing cells (FIG. 4b). FIG. 4b shows glucose-stimulated insulin secretion measured in the same experiments as in FIG. 4a. Next, the effect of VDAC1 inhibitors on ATP release was evaluated. FIG. 4c shows the effect of VDAC-antibody (VDAC1-ab, 10 nM), metformin (Met) or the VDAC1 blockers AKOS-022075291 (AKOS) and VBIT-4 (20 μM each) on ATP release after 1 h incubation at 1 mM glucose (1G) from INS-1 cells transfected with control plasmid or plVDAC1. As shown in FIG. 4c, the loss of cellular ATP was rapidly inhibited by metformin, VDAC1 antibody, AKOS or VBIT-4. ATP loss was substantiated in patch-clamp experiments in plVDAC1-expressing INS-1 cells, showing 30% higher membrane conductance than mtVDAC1-transfected cells (FIG. 4d). The increased conductance caused by plVDAC1 relative to control INS-1 cells was abolished by the acute addition of either VDAC1 antibody or metformin (FIG. 4e). Superfusion with metformin did not affect membrane currents in control INS-1 cells while abrogating the elevated conductance in plVDAC1-transformed cells (FIG. 4f). FIG. 4g shows that metformin (30 μM) blocks conductance of VDAC1 reconstituted into a planar lipid bilayer. Multi-channel recordings of VDAC1 conductance as a function of voltage with the average steady-state conductance of VDAC1 before (•) and 10-30 min after the addition of metformin (º). The effect of metformin average steady-state conductance at a given voltage (G) was normalized to the conductance at 10 mV (Go). FIG. 4h shows that the specific VDAC1 inhibitor VBIT-4 shows a similar effect as metformin. These results demonstrate that metformin at low, therapeutic concentrations, directly decreased VDAC1 conductance. Furthermore, the acute effect of metformin on VDAC1 solute permeation in intact cells is not mediated via activation of AMP kinase or through an antioxidant effect (FIG. 4i).

FIG. 4j-4k demonstrate that overexpression of plasma membrane VDAC1 (plVDAC1) causes cell death in INS-1 cells. FIG. 4j shows representative confocal images acquired from INS-1 cells transfected with mitochondrial VDAC1 (mtVDAC1) or plVDAC1 and cultured with either 5 mM glucose (5G) or 20 mM glucose (20G) for 24 h. Green (Calcein) and red (Ethidium homodimer-1, EthD1) indicate live and dead cells respectively. FIG. 4k shows average of ratios calculated by division of EthD1 intensity to calcein intensity.

### Example 4: Effect of Cysteine Depletion on VDAC1 Localization and Activity

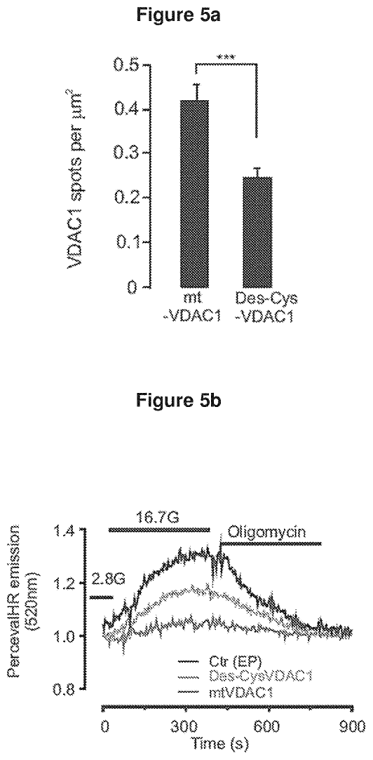

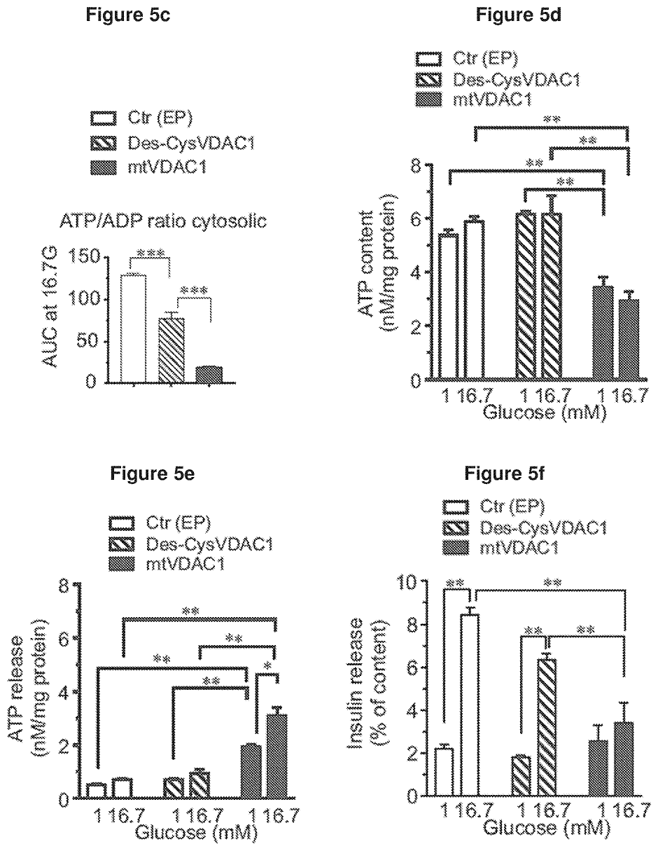

Cell surface mistargeting of VDAC1 in T2D may involve post-translational modification of its two cysteine residues (cys127/232) (Shoshan-Barmatz, V. et al, Molecular aspects of medicine 31, 227-285, 2010), although they are not important for VDAC1-induced apoptosis (Aram, L et al., 2010. Biochemical journal 427:445-454). Cysteine depleted VDAC1 was much more efficiently overexpressed in INS-1 cells than mtVDAC1, while the reciprocal suppression of VDAC2 observed with mtVDAC1 was absent. Despite the much higher expression, cysteine-depleted VDAC1 showed 50% less plasma membrane-near localization than mtVDAC1, as revealed by TIRF microscopy (FIG. 5a). Moreover, in contrast to mtVDAC1, the cellular ATP/ADP ratio and its increase by glucose stimulation were largely preserved (FIG. 5b, 5c), as was cellular ATP content (FIG. 5d). Furthermore, cysteine depleted VDAC1-expressing cells did not show increased ATP release, which was very pronounced in mtVDAC1 cells (FIG. 5e). The preserved cellular ATP content and ATP generation by glucose explain the near normal GSIS in the cells transfected with the mutant VDAC1 (FIG. 5f). These results are compatible with VDAC1 targeting to the β-cell plasma membrane by posttranslational cysteine modification, leading to ATP loss from the cells and impaired GSIS.

### Example 5: Blockade of Aberrantly Expressed VDAC1 Restores GSIS in T2D Islets and Pre-Diabetic Mice

FIG. 6 demonstrates that blockade of aberrantly expressed VDAC1 restores GSIS in T2D islets and pre-diabetic mice.

ATP levels in islets from hyperglycemic db/db mice were not raised by 16.7 mM glucose and there was increased ATP release (FIG. 6a, 6b). As expected from the VDAC1 surface localization, acute addition (1 h) of VDAC1 antibody or metformin reduced ATP release and increased its cellular levels (FIG. 6a, 6b). VDAC1 antibody or metformin increased insulin secretion in islets from hyperglycemic db/db mice at 16.7 mM glucose compared to control but had no effect in low glucose (FIG. 6c). Neither ATP content nor GSIS was affected by VDAC1 inhibition in islets from control mice.

Inhibition of cell membrane VDAC1 conductivity was further investigated in human islets. Inclusion of VDAC1 antibody or metformin in the culture medium prevented the attenuation of GSIS observed in the ND islets cultured at 20 mM glucose, while GSIS was unaffected after 5 mM glucose culture (FIG. 6d).

To probe for membrane effects of VDAC1 antibody, metformin, AKOS, or VBIT-4, the compounds were added to islets from 5 T2D donors and one IGT donor for 1 h and the results were pooled. VDAC1 inhibition increased islet ATP content both at 1 and 16.7 mM glucose. Notably, the ATP raising effect of glucose was 5-fold enhanced by VDAC1 inhibition (FIG. 6e). The T2D islets displayed severe blunting of GSIS, which was increased in parallel with the improved ATP generation by each of the four VDAC1 inhibitors (FIG. 6f). Inhibition of VDAC1 using metformin and VDAC1 antibody had no effect on cellular reductive capacity after culture at 5 mM glucose but markedly improved metabolism during glucotoxic conditions (FIG. 6g) or in T2D islets (FIG. 6h).

Thus, GSIS is markedly improved in T2D islets by the acute targeting of plasma membrane VDAC1, suggesting that this isoform rather than the decreased VDAC2 gene expression underlies the impaired beta cell function.

### Example 6: In Vivo Effect of the Specific VDAC1 Inhibitors

Islet cells from diabetic db/db mice were investigated. Like islets from T2D donors, β-cells from db/db but not C57/BL showed surface expression of VDAC1 (FIG. 7a, 7b). This was associated with increased VDAC1 exon1 mRNA, measured by qPCR (FIG. 7c). Neither ATP content nor GSIS was affected by VDAC1 inhibition in islets from control mice (FIG. 7d, 7e). Next, the effect of the VDAC1 inhibitor VBIT-4 was studied in vivo. Diabetes-prone young db/db mice were subjected to daily i.p injections of VBIT-4 from age 6 to 11 weeks. This treatment had no effects in the control C57/BL mice but prevented the development of the severe hyperglycemia, seen in vehicle-injected db/db mice. Upon VBIT-4 cessation, blood glucose concentrations rose gradually over several weeks, reaching those of vehicle-treated animals (FIG. 7f). The drug markedly improved glucose tolerance and GSIS (FIG. 7g, 7h, 7i, 7k, 7l). There was no effect on body weight (FIG. 7j). The early administration of VBIT-4 also prevented increases in water consumption and urine production in the db/db mice but had only marginal effects in 14-week old animals with severe diabetes and poluriea (data not shown).

Next, the effect of orally administered VBIT-4 on blood glucose in db/db mice was examined. VBIT-4 at the indicated doses or vehicle was administered by gavage to db/db mice from the age of 6-11 weeks. Blood was sampled and after for h fast once a week. Blood glucose was measured by a handheld device ContourXt (Bayer) using blood glucose strips. At the end of experiments (week 5) blood glucose was measured by the glucose-oxidase method (Infinity™ Glucose (Ox) TR15221 Thermo Scientific, USA. As seen in FIG. 8, daily oral administered VBIT-4 for 5 weeks reduced blood glucose in db/db mice.

### Example 7: Effect of a Racemic Mixture of VBIT-4 and its Enantiomers on Cellular ATP Content and Release

INS-1 cells were transiently transfected with a plasmid encoding plasma membrane targeted VDAC1 (pVDAC1) as described in the Material and Method section herein above and in Example 3 (see also Buettner et al 2000. PNAS 97:3201-3206). After transfection (24-32 h and 2-4 h recovery in normal culture medium, RPMI 1640 with complete supplement) the INS-1 cells were washed followed by preincubation in SAB-buffer (See Material and Method above) containing 2.8 mM glucose. The final 1 h incubation was performed in SAB-buffer containing 1 mM glucose and the VDAC1 inhibitory compounds to be tested. At the end, the cells were lysed using lysis buffer, sonicated and the lysate as well as the incubation medium were analyzed for ATP content using a commercially available kit (Biovision, K354-100). Cellular protein content was analyzed by Pierce™ BCA protein assay kit (Thermo Scientific, USA). Results presented in FIG. 9 show that racemic VBIT-4 as well as VBIT-4 enantiomer 1 (VBIT-4-1) and VBIT-4 enantiomer 2 (VBIT-4-2) efficiently inhibit ATP release and increase cellular ATP content. Apparently, racemic VBIT-4 potency to increase cellular ATP is intermediate between that of its enantiomers 1 and 2. There was no discernible difference in the inhibition of ATP release.

### Example 8: Interaction of VBIT-4 and Metformin with VDAC1

Microscale thermophoresis (MST) assay (Wienken C J et al., 2010. DOI: 10.1038/ncomms1093 Nature Comm. was conducted to examine whether VBIT-4 and/or metformin bind to VDCA1. Purified VDAC1 (162 nM), was labeled using the NanoTemper fluorescent protein-labeling Kit BLUE, and incubated with increasing concentrations of VBIT4 (0.625-100 mM) or metformin (1 to 100 mM). After 20 min of incubation, 3-5 ml of each sample were loaded into MST-grade glass capillaries, and the thermophoresis process was measured using the Monolith-NT115 apparatus. The results are presented as % of the bound fraction calculated as follows:

Fraction bound=100×(F−F min)/(F max−F min).

As is shown in FIG. 10, only VBIT-4, but not metformin, bound to VDAC1. These results suggest different modes of VDAC1 inhibition, with VBIT-4 binding and inhibiting VDAC1 while metformin inhibiting but not necessarily binding to VDCA1.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-48. (canceled)

49. A method for treating and/or preventing progression of diabetes in a subject in need thereof, comprising administering to the subject a therapeutically effective amount of at least one compound of general Formula (I1) or a pharmaceutical composition comprising same, wherein Formula (I1) is: 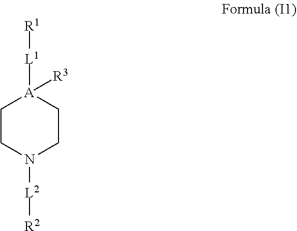
wherein:
A is carbon (C) or nitrogen (N);
R3 is absent, a hydrogen, an unsubstituted or substituted amide, or a heteroalkyl comprising 3-12 atoms (apart from hydrogen atoms), wherein at least one atom is a nitrogen, sulfur or oxygen atom, wherein when A is nitrogen (N), R3 is absent;
L1 is absent or is an amino linking group —NR4-, wherein R4 is hydrogen, a C1-5-alkyl, a C1-5-alkylene or a substituted alkyl —CH2R, wherein R is a functional group selected from the group consisting of hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl;
R1 is an aromatic moiety, which is optionally substituted with one or more of Z;
Z is independently at each occurrence a functional group selected from the group consisting of, hydrogen, halo, haloalkyl, haloalkoxy, perhaloalkoxy or C1-2-perfluoroalkoxy, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl and heteroaryl;
L2 is a linking group, such that when A is nitrogen (N), L2 is a group consisting of 4-10 atoms, apart from hydrogen atoms, optionally forming a ring, whereof at least one of the atoms is nitrogen, said nitrogen forming part of an amide group; and when A is carbon (C), then L2 is selected from C1-4 alkylene or a group consisting of 4-10 atoms, apart from hydrogen atoms, optionally forming a ring, whereof at least one of the atoms is nitrogen, said nitrogen forming part of an amide group;
R2 is a phenyl or a naphthyl, optionally substituted with halogen;
or an enantiomer, diastereomer, mixture or salt thereof.

50. The method according to claim 49, wherein A is nitrogen (N), and said linking group L2 is selected from the group consisting of a C4-6-alkylamidylene and a pyrrolidinylene, said linking group optionally substituted with one or two of alkyl, hydroxy, oxo or thioxo group.

51. The method according to claim 50, wherein L2 is selected from the group consisting of butanamidylene, N-methylbutanamidylene, N,N-dimethylbutanamidylene, 4-hydroxybutanamidylene, 4-oxobutanamidylene, 4-hydroxy-N-methylbutanamidylene, 4-oxo-N-methylbutanamidylene, 2-pyrrolidonyl, pyrrolidine-2,5-dionylene, 5-thioxo-2-pyrrolidinonylene and 5-methoxy-2-pyrrolidinonylene.

52. The method according to claim 51, wherein when L2 is butanamidylene, N-methylbutanamidylene, N,N-dimethylbutanamidylene, 4-hydroxybutanamidylene, 4-oxobutanamidylene, 4-hydroxy-N-methylbutanamidylene or 4-oxo-N-methylbutanamidylene, the carbon (C) in third position of the butanamide moiety is bonded to the nitrogen (N) of the piperazine ring and the nitrogen (N) of the butanamide moiety is bonded to R2; or wherein when L2 is 2-pyrrolidone, pyrrolidine-2,5-dione, 5-thioxo-2-pyrrolidone or 5-methoxy-2-pyrrolidone, a carbon (C) of the pyrrolidine moiety is bonded to the nitrogen (N) of the piperazine ring and the nitrogen (N) of the pyrrolidine moiety is bonded to R2.

53. The method according to claim 49, wherein A is carbon (C), R3 is a heteroalkyl group, and L2 is methylene.

54. The method according to claim 49, wherein the compound is of general Formula (Ia): 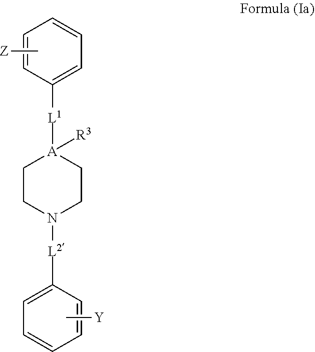
wherein:
A, R3, Z and L1 as defined in claim 49,
L2′ is a linking group selected from the group consisting of an C4-alkylamidylene, C5-alkylamidylene and C6-alkylamidylene, optionally substituted with one or two of alkyl, hydroxy, oxo or thioxo group; and
Y is halogen;
or an enantiomer, diastereomer, mixture or salt thereof.

55. The method according to claim 49, wherein the compound is of the general Formula (Ib): 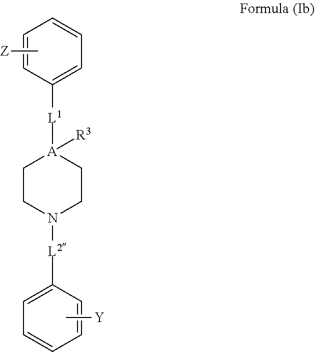
wherein:
A, R3, and Z are as defined in claim 49,
L1 is absent;
L2″ is a pyrrolidinylene linking group, optionally substituted with one or two of alkyl, hydroxy, oxo or thioxo group;
Y is halogen;
or an enantiomer, diastereomer, mixture or salt thereof.

56. The method according to claim 49, wherein the compound is of the general Formula (Ic): 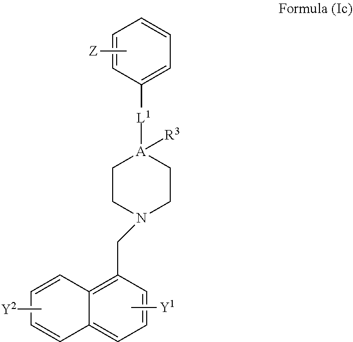
wherein:
A, R3, and Z are as defined in claim 49,
L1 is —NH—;
Y1 and Y2 are each independently absent or a halogen;
or an enantiomer, diastereomer, mixture or salt thereof.

57. The method according to claim 50, wherein the compound is of the general Formula (Id): 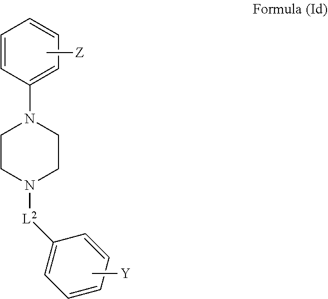
wherein
Z is C1-2-perfluoroalkoxy, and Y is halogen.

58. The method according to claim 57, wherein the compound is of structural Formula 1: 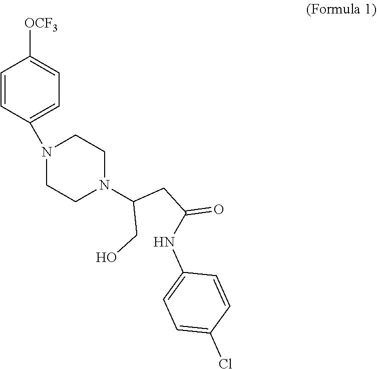 [SMILES: O=C(CC(CO)N1CCN(c2ccc(OC(F)(F)F)cc2)CC1)Nc1ccc(Cl)cc1]
or an enantiomer, diastereomer, mixture or salt thereof.

59. The method according to claim 58, wherein the compound is selected from the group consisting of a racemic mixture of Formula 1; an optically pure (+) enantiomer of Formula 1; and an optically pure (−) enantiomer of Formula 1.

60. The method according to claim 49, wherein the compound is of Formula (IIa): 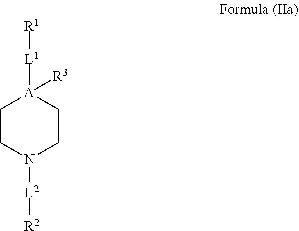
wherein:
A is carbon (C);
R3 is hydrogen or heteroalkyl chain comprising 3-12 atoms, apart from hydrogen atoms, wherein at least one is a heteroatom, selected from nitrogen, sulfur and oxygen;
L1 is an amino linking group —NR4-, wherein R4 is hydrogen, a C1-5-alkyl, a C1-5-alkylene or a substituted alkyl —CH₂R, wherein R is a functional group selected from hydrogen, halo, haloalkyl, cyano, nitro, hydroxyl, alkyl, alkenyl, aryl, alkoxyl, aryloxyl, aralkoxyl, alkylcarbamido, arylcarbamido, amino, alkylamino, arylamino, dialkylamino, diarylamino, arylalkylamino, aminocarbonyl, alkylaminocarbonyl, arylaminocarbonyl, alkylcarbonyloxy, arylcarbonyloxy, carboxyl, alkoxycarbonyl, aryloxycarbonyl, sulfo, alkylsulfonylamido, alkylsulfonyl, arylsulfonyl, alkylsulfinyl, arylsulfinyl or heteroaryl;
when R3 is heteroalkyl group comprising 3-12 atoms, apart from hydrogen atoms, then L1 forms a ring with R3;
R1 is an aromatic moiety, which is optionally substituted with one or more of C1-2-alkoxy, and/or C1-2-perfluoroalkoxy;
L2 is a linking group consisting of 4-10 atoms, apart from hydrogen atoms, optionally forming a ring, whereof at least one of the atoms is nitrogen, said nitrogen forming part of an amide group or L2 is C1-5 alkyl or C1-5 alkylene; said linking group L2 bonds piperidine or piperazine moiety at nitrogen (N) atom; and
R2 is an aryl, optionally substituted with halogen, optionally when R2 is a phenyl it is substituted with halogen, further optionally when R2 is naphthyl, L2 is an alkylenyl group;
or an enantiomer, diastereomer, mixture or salt thereof.

61. The method according to claim 60, wherein the compound of Formula (IIa) has the Formula 10:

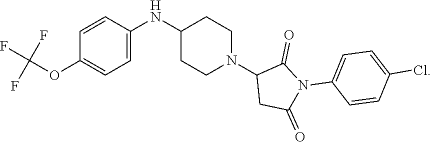 [SMILES: O=C1CC(N2CCC(Nc3ccc(OC(F)(F)F)cc3)CC2)C(=O)N1c1ccc(Cl)cc1]

62. The method according to claim 49, wherein preventing the progress of diabetes comprises preventing the progression of pre-diabetes to diabetes.

63. The method according to claim 49, wherein treating diabetes comprises at least one of inducing glucose-stimulated insulin secretion; improving glucose tolerance; restoring insulin secretion from pancreatic β-cells of the subject and prevention of β-cell dysfunction.

64. The method according to claim 49, wherein the compound or the pharmaceutical composition comprising same is administered in a route selected from the group consisting of oral administration, topical administration, parenteral administration, intranasal administration, administration by inhalation, or administration via a suppository.

65. A method for treating diabetes and/or preventing the progress of diabetes comprising administering to a subject in need thereof a therapeutically effective amount of a compound specifically binding to and inhibiting VDAC1 or a pharmaceutical composition comprising same, wherein the compound specifically binds to and inhibits VDAC1 expressed on pancreatic β-cells.

66. The method according to claim 65, wherein the compound inhibits ATP transport via the VDAC1.

67. The method according to claim 65, wherein the compound is an antibody specifically binding to VDAC1 expressed on diabetic β-cells and inhibiting ATP transport through the VDAC1.

68. A method for treating diabetes and/or preventing the progress of diabetes comprising administering to a subject in need thereof a therapeutically effective amount of a piperazine and/or piperidine derivative specifically binding to and inhibiting VDAC1 or a pharmaceutical composition comprising same, wherein the piperazine and/or piperidine derivative specifically binds to and inhibits VDAC1 expressed on pancreatic β-cells.
